SEQUENCE LISTING (1 sequence)

SEQ ID NO: 1 - DNA, 1285 bp, Artificial sequence
  human G6PC promoter -1227/+57
gagctcaggaattcaagaccagcctgggcaacatggaaaaaccccatctctacaaaagatagaaaaattagccaggcatggtggcgtgtgcctgtggtcccagctactcaggaggctgaggtgggaggatcacattagcccaggaggttgaggctgcagtgagccgtgattatgccactgcactccagcctgggagacagagtgagaccctgtttcaaaaaaaagagagagaaaatttaaaaaagaaaacaacaccaagggctgtaactttaaggtcattaaatgaattaatcactgcattcaaaaacgattactttctggccctaagagacatgaggccaataccaggaagggggttgatctcccaaaccagaggcagaccctagactctaatacagttaaggaaagaccagcaagatgatagtccccaatacaatagaagttactatattttatttgttgtttttcttttgttttgttttgttttgttttgttttgttttagagactggggtcttgctcgattgcccaggctgtagtgcagcggtgggacaatagctcactgcagactccaactcctgggctcaagcaatcctcctgcctcagcctcctgaatagctgggactacaagggtacaccatcacacacaccaaaacaattttttaaatttttgtgtagaaacgagggtcttgctttgttgcccaggctggtctccaactcctggcttcaagggatcctcccacctcagcctcccaaattgctgggattacaggtgtgagccaccacaaccagccagaactttactaattttaaaattaagaacttaaaacttgaatagctagagcaccaagatttttctttgtccccaaataagtgcagttgcaggcatagaaaatctgacatctttgcaagaatcatcgtggatgtagactctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcagagcaatcaccaccaagcctggaataactgcaagggctctgctgacatcttc

(19) United States
(12) Patent Application Publication
(10) Pub. No.: US 2022/0026414 A1
(43) Pub. Date: Jan. 27, 2022

(54) CELL-BASED BIOIDENTITY TEST FOR INSULIN

(71) Applicant: Merck Sharp & Dohme Corp., Rahway, NJ (US)

(72) Inventor: Junming Yie, Warren, NJ (US)

(73) Assignee: Merck Sharp & Dohme Corp., Rahway, NJ (US)

(21) Appl. No.: 17/299,034

(22) PCT Filed: Dec. 6, 2019
(86) PCT No.: PCT/US2019/064814

§ 371 Date: Jun. 2, 2021

Related U.S. Application Data
(60) Provisional application No. 62/778,563, filed on Dec. 12, 2018.

(51) Int. Cl. G01N 33/50; C12N 15/867; C12N 9/02

(52) U.S. Cl. CPC G01N 33/5067; C12N 15/867; C12Y 113/12; G01N 2333/62; C12N 9/0069

(57) ABSTRACT

A functional cell-based assay for use as a bioidentity assay for insulin or insulin analogs is described. The assay may be used as a replacement of the rabbit blood sugar method disclosed in USP<121> Insulin Assays.

### BACKGROUND OF THE INVENTION

### (1) Field of the Invention

The present invention relates to a functional cell-based assay for use as a bioidentity assay for insulin or insulin analogs. The assay may be used as a replacement of the rabbit blood sugar method disclosed in USP<121> Insulin Assays.

### (2) Description of Related Art

Insulin and its analogs represent a significant market share in both type I and type II diabetes treatments. Currently, the global annual insulin market is around $17.5 billion US dollars. Regulatory agencies such as the United States Food and Drug Administration (U.S. FDA) require product batches to undergo and pass a bioidentity test prior to release of the product to the U.S. market. In the United States, the bioidentity test for determining the potency of insulin and insulin analogs is set forth in Chapter “<121> Insulin Assays” of the United States Pharmacopeia (USP<121>), which mandates a rabbit blood sugar test for the potency evaluation of insulin and insulin analogs. However, as part of USP's ongoing effort to phase out animal-based assays in favor of modern in vitro testing, a replacement cell-based bioidentity test was allowed for use in bioidentity tests for insulin glargine and insulin lispro. In the future, the USP plans to omit the rabbit method completely but only after multiple manufacturers have been able to demonstrate that the cell-based method is appropriate for all their insulin products. The USP has encouraged manufacturers to contact USP if the new in vitro test is not suitable for their insulin products or if they would like to submit an alternative validated and approved in vitro test for this purpose.

The use of the rabbit-based bioidentity test for insulin and insulin analogs has existed for decades prior to the development of reliable physicochemical test methods based on high performance liquid chromatography (HPLC). Physicochemical assays based on HPLC assays to determine the content of insulin and insulin analogs are much more precise and accurate than the rabbit blood sugar method. In the spirit of the 3R principles (reduction, refinement, and replacement) in animal experiments, most pharmacopeias, including Europe, Japan, India, and more, have forgone testing in living animals in lieu of the physicochemical tests. However, in the U.S, the bioidentity test in USP <121> is still required by the U.S. FDA since the bioidentity test forms a part of the quality specifications for insulin drug substances for the U.S. market.

The need to reduce animal use in insulin bioassays has long been recognized (Trethewey, J. Pharma. Biomed. Anal. 7:189-197 (1989)) but the a bioidentity assay is required to determine the biological activity of insulin drug substance. The International Conference on Harmonization (ICH) guideline Q6B (Specifications: Test Procedures and Acceptance Criteria for Biotechnological/biological Products, Q6B ICH Harmonised Tripartite Guideline, Specifications: Test Procedures And Acceptance Criteria For Biotechnological/Biological Products Q6B (1999)) states that the higher-order structure of complex molecules cannot be confirmed by extensive physicochemical information but can be inferred from the biological activity. Even though insulin and its analogs fall between small molecules and common large molecules like proteins and antibodies, it is prudent to have a bioidentity assay in place to infer biological activity of the drug substance. In light of the desire to reduce animal use in insulin bioassays, a functional cell-based in vitro assay seems to be an appropriate way to replace the animal based bioidentity test since it can measure the biological activity of the insulin or insulin analog in a physiologically relevant setting.

Recently, an in vitro test for insulin glargine, which measures pIR in CHO-IR cells was reported by Tennagels et al. (Stimuli to the Revision Process: A Proposed In Vitro Cell Based Bioassay to Include into the USP general chapter Insulin Assays <121>, U.S. Pharmacopoeial Forum 43(4) (2017)). In a comparison study, the pIR in vitro assay was shown to provide similar results as the rabbit blood sugar identity test (Ibid.) and was approved by the U.S. FDA to be used as an alternative to the rabbit blood sugar method (Hack et al., Progress towards the replacement of the rabbit blood sugar bioidentity assay by an in vitro test for batch release of insulins in quality control, Altex 34(4): 565-566 (2017)).

The current cell-based pIR in vitro assay disclosed in the USP<121> revision is an antibody-based method that uses antibodies that bind phosphotyrosines to measure the phosphorylation of the tyrosine residues on the insulin receptor expressed on the surface of recombinant host cells genetically engineered to express the insulin receptor upon exposure of the recombinant host cells to insulin (see also Hack et al. op. cit.). The pIR in vitro assay requires the use of two antibodies, a first antibody that binds phosphotyrosines and a second antibody that binds the first antibody and is labeled with a detection moiety. Accurate results using the pIR in vitro assay are dependent on adequately lysing the cells to release phosphorylated insulin receptor and the assiduousness of the washes following each antibody binding step. While the assay may be useful as a bioidentity test, because of the multiple washing steps, the assay is time and labor intensive and as discussed herein, produces about 20% variability. Thus, there is a need for an alternative bioidentity test.

### BRIEF SUMMARY OF THE INVENTION

The present invention provides a cell-based bioidentity assay for determining the potency of insulin and insulin analogs comprising measuring expression of a reporter gene under the control of a glucose-6-phosphate catalytic-subunit-encoding gene C (G6PC) promoter transfected into a hepatic host cell that comprises a gluconeogenesis pathway following contact of a culture of the cells with an insulin or insulin analog. In cells comprising a gluconeogenesis pathway (See FIG. 5), the G6PC promoter is downregulated by insulin; therefore, the assay measures the decrease in expression of the reporter gene when the host cell is exposed to insulin. The decrease in expression may be measured using a detectable substrate for the reporter molecule encoded by the reporter gene. For example, the reporter gene may encode an enzyme such as luciferase and expression of the luciferase may be detected by providing a luciferin substrate to the cell culture that fluoresces when cleaved by luciferase. In the assay, the potency of the insulin or insulin analog is inversely proportional to the decrease in expression of the reporter gene. Unlike the in vitro pIR assay disclosed in the USP<121> revision, the assay does not require use of antibodies and the washes they entail for detecting the presence and accumulation of a molecule in response to treatment with the insulin or insulin analog.

In a further embodiment, the present invention provides a method for detecting the presence of an insulin or insulin analog in a sample comprising (a) providing a cell culture of recombinant cells capable of gluconeogenesis, wherein the recombinant cells comprise a nucleic acid molecule encoding a reporter molecule operably linked to the promoter for the glucose-6-phosphate catalytic-subunit-encoding gene C (G6PC); (b) mixing an aliquot of the cell culture with a cell culture medium comprising the insulin or insulin analog to provide an assay cell culture and incubating the assay cell culture for about 15 to 27 hours; and (c) detecting the presence or amount of the reporter molecule in the assay cell culture, wherein a decrease in the amount of reporter molecule relative to the amount of the reporter molecule in a control cell culture comprising the recombinant cells in a medium lacking the insulin or insulin analog and incubated for about 15 to 27 hours indicates the presence of the insulin or insulin analog in the sample.

In particular embodiments of this portion of the invention, the promoter is a human G6PC promoter comprising the nucleotide sequence set forth SEQ ID NO: 1. In a further embodiment, the reporter molecule is an enzyme and a substrate for the enzyme is provided to the culture to detect the reporter molecule. In a further embodiment, the enzyme is luciferase and the substrate is luciferin.

In a particular embodiment of this portion of the invention, the cell culture medium comprises at least 1.5 g/L of glucose. In a further embodiment, the cell culture medium comprises about 4.5 g/L of glucose. In a particular embodiment, the cell culture medium comprises at least 5% fetal bovine serum. In a further embodiment, the cell culture medium comprises about 10% fetal bovine serum. In a further embodiment, the cell culture medium comprises at least 1.5 g/L glucose and at least 5% fetal bovine serum. In a further embodiment, the cell culture medium comprises at least 1.5 g/L glucose and about 10% fetal bovine serum. In a further embodiment, the cell culture medium comprises about 4.5 g/L glucose and at least 5% fetal bovine serum. In a further embodiment, the cell culture medium comprises about 4.5 g/L glucose and about 10% fetal bovine serum.

In a particular embodiment of this portion of the invention, the cell culture is incubated for about 17±1 hours in the presence of the insulin or insulin analog prior to detecting the expression of the reporter molecule.

In a further embodiment, the present invention provides a method for determining the potency of an insulin or insulin analog in a sample comprising (a) providing a cell culture of recombinant cells capable of gluconeogenesis, wherein the recombinant cells comprise a nucleic acid molecule encoding a reporter molecule operably linked to the promoter for the glucose-6-phosphate catalytic-subunit-encoding gene C (G6PC); (b) mixing equal aliquots of the cell culture with a series of dilutions of cell culture medium comprising the insulin or insulin analog to provide a series of assay cell cultures and incubating the series of assay cell cultures for about 15 to 27 hours; (c) detecting the reporter molecule in the series of assay cell cultures to provide data for the assay cell culture and generating a dose response curve from the data; and (d) comparing the dose response curve to a reference dose response curve obtained from one or more series of reference assay cell cultures determined by a method comprising (i) mixing equal aliquots of the cell culture with a series of dilutions of a reference cell culture medium comprising a known amount of the insulin or insulin analog to provide a series of reference assay cell cultures, (ii) incubating the series of reference assay cell cultures for about 15 to 27 hours, and (iii) detecting the reporter molecule in the series of reference assay cell cultures to provide data for the reference assay cell culture and generating the reference dose response curve from the data, to provide the potency of the insulin or insulin analog in the sample.

In particular embodiments of this portion of the invention, the series of dilutions comprises a seven-fold serial dilution. In further embodiments, the sample series of assays and the reference series of assays are prepared in duplicate. In further embodiments, the sample series of assays are prepared in triplicate and the reference series of assays are prepared in duplicate.

In particular embodiments of this portion of the invention, the assay includes a control cell culture assay comprising incubating the recombinant cells in a medium lacking the sample for about 15 to 27 hours.

In particular embodiments of this portion of the invention, the promoter is a human G6PC promoter comprising the nucleotide sequence set forth SEQ ID NO: 1. In a further embodiment, the reporter molecule is an enzyme and a substrate for the enzyme is provided to the culture to detect the reporter molecule. In a further embodiment, the enzyme is luciferase and the substrate is luciferin.

In a particular embodiment of this portion of the invention, the cell culture medium comprises at least 1.5 g/L of glucose. In a further embodiment, the cell culture medium comprises about 4.5 g/L of glucose. In a particular embodiment, the cell culture medium comprises at least 5% fetal bovine serum. In a further embodiment, the cell culture medium comprises about 10% fetal bovine serum. In a further embodiment, the cell culture medium comprises at least 1.5 g/L glucose and at least 5% fetal bovine serum. In a further embodiment, the cell culture medium comprises at least 1.5 g/L glucose and about 10% fetal bovine serum. In a further embodiment, the cell culture medium comprises about 4.5 g/L glucose and at least 5% fetal bovine serum. In a further embodiment, the cell culture medium comprises about 4.5 g/L glucose and about 10% fetal bovine serum.

In a particular embodiment of this portion of the invention, the cell culture is incubated for about 17±1 hours in the presence of the insulin or insulin analog prior to detecting the expression of the reporter molecule.

In a further embodiment, the present invention provides a method for determining the bioidentity of an insulin or insulin analog in a sample comprising (a) providing a cell culture of recombinant cells capable of gluconeogenesis, wherein the recombinant cells comprise a nucleic acid molecule encoding a reporter molecule operably linked to the promoter for the glucose-6-phosphate catalytic-subunit-encoding gene C (G6PC); (b) mixing equal aliquots of the cell culture with a series of dilutions of cell culture medium comprising the insulin or insulin analog to provide a series of assay cell cultures and incubating the series of assay cell cultures for about 15 to 27 hours; (c) detecting the reporter molecule in the series of assay cell cultures to provide data for the assay cell culture and generating a dose response curve from the data; and (d) comparing the dose response curve to a reference dose response curve obtained from one or more series of reference assay cell cultures determined by a method comprising (i) mixing equal aliquots of the cell culture with a series of dilutions of a reference cell culture medium comprising a known amount of the insulin or insulin analog to provide a series of reference assay cell cultures, (ii) incubating the series of reference assay cell cultures for about 15 to 27 hours, and (iii) detecting the reporter molecule in the series of reference assay cell cultures to provide data for the reference assay cell culture and generating the reference dose response curve from the data, to provide the bioidentity of the insulin or insulin analog in the sample.

In particular embodiments of this portion of the invention, the series of dilutions comprises a seven-fold serial dilution. In further embodiments, the sample series of assays and the reference series of assays are prepared in duplicate. In further embodiments, the sample series of assays are prepared in triplicate and the reference series of assays are prepared in duplicate.

In particular embodiments of this portion of the invention, the assay includes a control cell culture assay comprising incubating the recombinant cells in a medium lacking the sample for about 15 to 27 hours.

In particular embodiments of this portion of the invention, the promoter is a human G6PC promoter comprising the nucleotide sequence set forth SEQ ID NO: 1. In a further embodiment, the reporter molecule is an enzyme and a substrate for the enzyme is provided to the culture to detect the reporter molecule. In a further embodiment, the enzyme is luciferase and the substrate is luciferin.

In a particular embodiment of this portion of the invention, the cell culture medium comprises at least 1.5 g/L of glucose. In a further embodiment, the cell culture medium comprises about 4.5 g/L of glucose. In a particular embodiment, the cell culture medium comprises at least 5% fetal bovine serum. In a further embodiment, the cell culture medium comprises about 10% fetal bovine serum. In a further embodiment, the cell culture medium comprises at least 1.5 g/L glucose and at least 5% fetal bovine serum. In a further embodiment, the cell culture medium comprises at least 1.5 g/L glucose and about 10% fetal bovine serum. In a further embodiment, the cell culture medium comprises about 4.5 g/L glucose and at least 5% fetal bovine serum. In a further embodiment, the cell culture medium comprises about 4.5 g/L glucose and about 10% fetal bovine serum.

In a particular embodiment of this portion of the invention, the cell culture is incubated for about 17±1 hours in the presence of the insulin or insulin analog prior to detecting the expression of the reporter molecule.

The present invention further provides a method for releasing a manufacturing batch or lot of drug product comprising insulin or insulin analog, comprising (a) providing a manufacturing batch or lot of a drug product; (b) obtaining a sample from the batch or lot and subjecting the sample to a cell-based bioidentity test to determine whether the batch or lot can be released, wherein the bioidentity test comprises (i) mixing equal aliquots of the cell culture with a series of dilutions of cell culture medium comprising the sample to provide a series of assay cell cultures and mixing equal aliquots of the cell culture with a series of dilutions of a reference cell culture medium comprising a known amount of the insulin or insulin analog to provide a series of reference assay cell cultures; (ii) incubating the series of assay cell cultures and the series of reference cell cultures for about 15 to 27 hours; (iii) detecting the reporter molecule in the series of assay cell cultures to provide data for the assay cell culture and generating a sample dose response curve from the data and detecting the reporter molecule in the series of reference assay cell cultures to provide data for the reference assay cell culture and generating a reference dose response curve from the data; and (c) comparing the sample dose response curve to the reference dose response curve and releasing the batch or lot when the difference between the sample dose response curve is within a predetermined amount from the reference dose response curve.

In particular embodiments of this portion of the invention, the series of dilutions comprises a seven-fold serial dilution. In further embodiments, the sample series of assays and the reference series of assays are prepared in duplicate. In further embodiments, the sample series of assays are prepared in triplicate and the reference series of assays are prepared in duplicate.

In particular embodiments of this portion of the invention, the assay includes a control cell culture assay comprising incubating the recombinant cells in a medium lacking the sample for about 15 to 27 hours.

In particular embodiments of this portion of the invention, the recombinant cells are H4IIE rat hepatoma cells or HepG2 human hepatoma cells, which comprise the nucleic acid molecule encoding the reporter molecule operably linked to the promoter for the glucose-6-phosphate catalytic-subunit-encoding gene C (G6PC). In further embodiments, the recombinant cells are H4IIE rat hepatoma cells or HepG2 human hepatoma cells in which the nucleic acid molecule encoding the reporter molecule operably linked to the promoter for the glucose-6-phosphate catalytic-subunit-encoding gene C (G6PC) has been integrated into the genome of the recombinant cells.

In particular embodiments of this portion of the invention, the promoter is a human G6PC promoter comprising the nucleotide sequence set forth SEQ ID NO: 1. In a further embodiment, the reporter molecule is an enzyme and a substrate for the enzyme is provided to the culture to detect the reporter molecule. In a further embodiment, the enzyme is luciferase and the substrate is luciferin.

In particular embodiments of this portion of the invention, the cell culture medium comprises at least 1.5 g/L of glucose. In a further embodiment, the culture medium comprises about 4.5 g/L of glucose. In a particular embodiment, the cell culture medium comprises at least 5% fetal bovine serum. In a further embodiment, the cell culture medium comprises about 10% fetal bovine serum. In a further embodiment, the cell culture medium comprises at least 1.5 g/L glucose and at least 5% fetal bovine serum. In a further embodiment, the cell culture medium comprises at least 1.5 g/L glucose and about 10% fetal bovine serum. In a further embodiment, the cell culture medium comprises about 4.5 g/L glucose and at least 5% fetal bovine serum. In a further embodiment, the cell culture medium comprises about 4.5 g/L glucose and about 10% fetal bovine serum.

In particular embodiments of this portion of the invention, the cell culture is incubated for about 17±1 hours in the presence of the insulin or insulin analog prior to detecting the expression of the reporter molecule.

The present invention further provides a method for determining the potency of an insulin or insulin analog comprising (a) providing a cell culture of recombinant cells capable of gluconeogenesis comprising a nucleic acid molecule encoding a reporter molecule operably linked to the promoter for the glucose-6-phosphate catalytic-subunit-encoding gene C (G6PC); (b) mixing an aliquot of the cell culture with a cell culture medium comprising the insulin or insulin analog to provide an assay cell culture and incubating the assay cell culture for 15 to 27 hours; and (c) detecting the presence of the reporter molecule in the assay cell culture, wherein a decrease in reporter molecule relative to amount of reporter molecule in a control cell culture comprising the recombinant cells in a medium lacking the insulin or insulin analog and incubated for the 15 to 27 hours, determines the potency of the insulin or insulin analog.

In particular embodiments of this portion of the invention, step (b) comprises mixing equal aliquots of the cell culture with a series of dilutions of cell culture medium comprising the insulin or insulin analog to provide a series of assay cell cultures and incubating the series of assay cell cultures for 15 to 27 hours.

In particular embodiments of this portion of the invention, step (b) comprises mixing equal aliquots of the cell culture with a series of dilutions of cell culture medium comprising the insulin or insulin analog to provide a series of assay cell cultures and mixing equal aliquots of the cell culture with a series of dilutions of a reference cell culture medium comprising a known amount of the insulin or insulin analog to provide a series of reference assay cell cultures, each of which are incubated for 15 to 27 hours and step (c) comprises detecting the reporter molecule in the series of assay cell cultures to provide data for the assay cell culture and generating a sample dose response curve from the data and detecting the reporter molecule in the series of reference assay cell cultures to provide data for the reference assay cell culture and generating a reference dose response curve from the data; and ins a step (d) comparing the sample dose response curve to the reference dose response curve to determine the potency of the insulin or insulin analog.

In particular embodiments of this portion of the invention, the recombinant cells are H4IIE rat hepatoma cells or HepG2 human hepatoma cells, which comprise the nucleic acid molecule encoding the reporter molecule operably linked to the promoter for the glucose-6-phosphate catalytic-subunit-encoding gene C (G6PC). In further embodiments, the recombinant cells are H4IIE rat hepatoma cells or HepG2 human hepatoma cells in which the nucleic acid molecule encoding the reporter molecule operably linked to the promoter for the glucose-6-phosphate catalytic-subunit-encoding gene C (G6PC) has been integrated into the genome of the recombinant cells.

In particular embodiments of this portion of the invention, the promoter is a human G6PC promoter comprising the nucleotide sequence set forth SEQ ID NO: 1. In a further embodiment, the reporter molecule is an enzyme and a substrate for the enzyme is provided to the culture to detect the reporter molecule. In a further embodiment, the enzyme is luciferase and the substrate is luciferin.

In particular embodiments of this portion of the invention, the cell culture medium comprises at least 1.5 g/L of glucose. In a further embodiment, the culture medium comprises about 4.5 g/L of glucose. In a particular embodiment, the cell culture medium comprises at least 5% fetal bovine serum. In a further embodiment, the cell culture medium comprises about 10% fetal bovine serum. In a further embodiment, the cell culture medium comprises at least 1.5 g/L glucose and at least 5% fetal bovine serum. In a further embodiment, the cell culture medium comprises at least 1.5 g/L glucose and about 10% fetal bovine serum. In a further embodiment, the cell culture medium comprises about 4.5 g/L glucose and at least 5% fetal bovine serum. In a further embodiment, the cell culture medium comprises about 4.5 g/L glucose and about 10% fetal bovine serum.

The present invention further provides recombinant cell capable of gluconeogenesis comprising a nucleic acid molecule encoding a reporter molecule operably linked to the promoter for the glucose-6-phosphate catalytic-subunit-encoding gene C (G6PC).

In particular embodiments of this portion of the invention, the recombinant cells are H4IIE rat hepatoma cells or HepG2 human hepatoma cells, which comprise the nucleic acid molecule encoding the reporter molecule operably linked to the promoter for the glucose-6-phosphate catalytic-subunit-encoding gene C (G6PC). In further embodiments, the recombinant cells are H4IIE rat hepatoma cells or HepG2 human hepatoma cells in which the nucleic acid molecule encoding the reporter molecule operably linked to the promoter for the glucose-6-phosphate catalytic-subunit-encoding gene C (G6PC) has been integrated into the genome of the recombinant cells.

In particular embodiments of this portion of the invention, the promoter is a human G6PC promoter comprising the nucleotide sequence set forth SEQ ID NO: 1. In a further embodiment, the reporter molecule is an enzyme, which in a further embodiment is luciferase.

The present invention further provides a cell culture comprising a medium and a recombinant cell capable of undergoing gluconeogenesis comprising a nucleic acid molecule encoding a reporter molecule operably linked to the promoter for the glucose-6-phosphate catalytic-subunit-encoding gene C (G6PC).

In particular embodiments of this portion of the invention, the recombinant cells are H4IIE rat hepatoma cells or HepG2 human hepatoma cells, which comprise the nucleic acid molecule encoding the reporter molecule operably linked to the promoter for the glucose-6-phosphate catalytic-subunit-encoding gene C (G6PC). In further embodiments, the recombinant cells are H4IIE rat hepatoma cells or HepG2 human hepatoma cells in which the nucleic acid molecule encoding the reporter molecule operably linked to the promoter for the glucose-6-phosphate catalytic-subunit-encoding gene C (G6PC) has been integrated into the genome of the recombinant cells.

In particular embodiments of this portion of the invention, the promoter is a human G6PC promoter comprising the nucleotide sequence set forth SEQ ID NO: 1. In a further embodiment, the reporter molecule is an enzyme, which in a further embodiment is luciferase.

The present invention further provides a cell culture comprising a medium and a recombinant cell capable of gluconeogenesis comprising a nucleic acid molecule encoding a reporter molecule operably linked to the promoter for the glucose-6-phosphate catalytic-subunit-encoding gene C (G6PC).

In particular embodiments of this portion of the invention, the recombinant cells are H4IIE rat hepatoma cells or HepG2 human hepatoma cells, which comprise the nucleic acid molecule encoding the reporter molecule operably linked to the promoter for the glucose-6-phosphate catalytic-subunit-encoding gene C (G6PC). In further embodiments, the recombinant cells are H4IIE rat hepatoma cells or HepG2 human hepatoma cells in which the nucleic acid molecule encoding the reporter molecule operably linked to the promoter for the glucose-6-phosphate catalytic-subunit-encoding gene C (G6PC) has been integrated into the genome of the recombinant cells.

In particular embodiments of this portion of the invention, the promoter is a human G6PC promoter comprising the nucleotide sequence set forth SEQ ID NO: 1. In a further embodiment, the reporter molecule is an enzyme, which in a further embodiment is luciferase.

In particular embodiments of this portion of the invention, the cell culture medium comprises at least 1.5 g/L of glucose. In a further embodiment, the culture medium comprises about 4.5 g/L of glucose. In a particular embodiment, the cell culture medium comprises at least 5% fetal bovine serum. In a further embodiment, the cell culture medium comprises about 10% fetal bovine serum. In a further embodiment, the cell culture medium comprises at least 1.5 g/L glucose and at least 5% fetal bovine serum. In a further embodiment, the cell culture medium comprises at least 1.5 g/L glucose and about 10% fetal bovine serum. In a further embodiment, the cell culture medium comprises about 4.5 g/L glucose and at least 5% fetal bovine serum. In a further embodiment, the cell culture medium comprises about 4.5 g/L glucose and about 10% fetal bovine serum. In a further embodiment, the cell culture comprises an insulin or insulin analog.

The present invention further provides a nucleic acid molecule encoding a reporter molecule operably linked to the promoter for the glucose-6-phosphate catalytic-subunit-encoding gene C (G6PC). In particular embodiments of this portion of the invention, the promoter is a human G6PC promoter comprising the nucleotide sequence set forth SEQ ID NO: 1. In a further embodiment of this portion of the invention, the reporter molecule is an enzyme, which in a further embodiment is luciferase.

The present invention further provides a vector comprising the above nucleic acid molecule. In particular embodiments of this portion of the invention, the vector is a plasmid or recombinant virus. In a further embodiment, the recombinant virus is a lentivirus.

### BRIEF DESCRIPTION OF THE DRAWINGS

FIG. 1A shows a diagram of assay mechanism of action. A nucleic acid molecule encoding luciferase operably linked to the G6PC promoter was integrated into the genome of H4IIE rat hepatocytes using lentivirus transduction. When insulin binds to the insulin receptor on the cell membrane, it induces a series of signal transduction events culminating in the downregulation of G6PC promoter activity and expression of the luciferase. Also shown is a general protocol for practicing the present invention.

FIG. 1B-1 and FIG. 1B-2 show the results of an initial screening of the 65 clones. Fold changes are the responses normalized by the readout of 1 nM insulin from each clone.

FIG. 1C shows full dose responses of insulin with clones 17 and 58 as well as the stable pool. The plot uses fold changes, which are the responses normalized by the maximum responses of insulin from each clone.

FIG. 2A shows an H4IIE G6P-Luc reporter assay run using clone C17-2 with different treatment times: 7-hour, 17-hour, and 24-hour treatment times.

FIG. 2B shows the effects different amounts of glucose in DMEM of the assay medium had on the H4IIE G6P-Luc reporter assay run using clone C17-2. DMEM with high glucose (4.5 g/L), low glucose (1.5 g/L), or no glucose was used.

FIG. 2C shows the effects different amount of FBS or with 1% BSA in the assay medium had on the H4IIE G6P-Luc reporter assay run using clone C17-2. Different amounts of FBS, including 10%, 5%, 2%, and 1% FBS, were used. Since different amount of FBS or BSA had various luciferase readouts: fold changes, which are the responses normalized by the maximum responses of insulin from each condition, were used in the plot to simplify the view.

FIG. 2D shows a dose response curve of the assay using old passage (P29) of H4IIE_G6P-Luc_C17-2 stable cell line.

FIG. 3A shows the testing scheme of the pre-qualification study for H4IIE G6P-Luc reporter assay run using clone C17-2.

FIG. 3B shows a representative graph of the dose-response curve from the pre-qualification study for H4IIE G6P-Luc reporter assay run using clone C17-2.

FIG. 3C shows relative potency data points grouped by potency levels and analysts for H4IIE G6P-Luc reporter assay run using clone C17-2.

FIG. 3D shows a plot of % Relative Bias with two-sided 95% confidence interval for H4IIE G6P-Luc reporter assay run using clone C17-2.

FIG. 3E shows a linearity plot of the pre-qualification study for H4IIE G6P-Luc reporter assay run using clone C17-2. The natural log (LN) values of expected relative potencies (X axis) and LN values of observed relative potencies (Y-axis) are plotted.

FIGS. 4A, 4B, 4C, and 4D show assay acceptance limits from available data set for the H4IIE G6P-Luc reporter assay run using clone C17-2. FIG. 4A: Plot of Slope ratio; FIG. 4B: Plot of % A difference; FIG. 4C: Plot of % D difference; FIG. 4D: Plot of A/D ratio.

FIG. 5 shows the gluconeogenesis pathway present in liver cells and the insulin signaling pathway in such liver cells. Insulin induces auto-phosphorylation of the insulin receptor (IR) to phosphorylated IR (p-IR), which begins a cascade of intracellular events that in liver cells includes downregulation of expression of the G6PC gene, thereby, inhibiting conversion of glucose-6-phosphate to glucose. The prior art Phosphorylated Insulin Receptor assay measures the auto-phosphorylation of the insulin receptor (p-IR) in response to exogenously added insulin. In contrast, the assay of the present invention, the G6PC-Reporter assay, measures the decrease of reporter gene expression regulated by the G6PC promoter in response to exogenously added insulin. PI3K is phosphatidylinositol-3-kinase; AKT is protein kinase B; PEPCK is Phosphoenolpyruvate carboxykinase.

FIG. 6 shows a representative example of a typical seven-fold dilution.

FIG. 7 shows a seven-fold serial dilutions along with a control that contains no insulin or insulin analog arranged on a 96-well plate.

FIG. 8 shows a representative dose response curve.

### DETAILED DESCRIPTION OF THE INVENTION

Insulin lowers blood glucose level via two main mechanisms, lowering liver glucose output and promoting glucose uptake in peripheral tissues like muscle and fat tissue. In the liver, glucose can be generated through either de novo glucose synthesis, or gluconeogenesis, or the breakdown of glycogen, or glycogenolysis, in which the gluconeogenesis pathway plays a major role in glucose production, especially in the pathogenesis of type 2 diabetes (Saltiel, New perspectives into the molecular pathogenesis and treatment of type 2 diabetes, Cell 104(4):517-529 (2001); Petersen et al., Mechanisms of Insulin Action and Insulin Resistance, Physiol. Rev. 98: 2133-2223 (2018)). Glucose-6-phosphatase (G6Pase) and phosphoenolpyruvate carboxykinase (PEPCK) are two critical enzymes in the gluconeogenesis pathway that are suppressed by insulin (Barthel et al., Novel concepts in insulin regulation of hepatic gluconeogenesis, Am. J. Physiol. 285: E685-692 (2003)). PEPCK catalyzes one rate-limiting step of gluconeogenesis, the reaction of oxaloacetic acid to phosphoenolpyruvate, whereas G6Pase catalyzes the reaction of glucose-6-phosphate to free glucose, which is the final step of gluconeogenesis. It is known that the expression of the catalytic subunit of G6Pase (G6PC) and PEPCK are suppressed by insulin through transcriptional regulation, and the promoter regions of G6PC and PEPCK have been well characterized (Schmoll et al., Cloning and sequencing of the 5′ region of the human glucose-6-phosphatase gene: transcriptional regulation by cAMP, insulin and glucocorticoids in H4IIE hepatoma cells, FEBS Letts. 383:63-66 (1996); Iynedjian et al., Glucokinase and cytosolic phosphoenolpyruvate carboxykinase (GTP) in the human liver. Regulation of gene expression in cultured hepatocytes, J. Clin. Invest. 95: 1966-1973 (1995)). Between the two, G6PC is the more significantly regulated gene by insulin, and its promoter region has been well characterized and extensively studied (Schmoll op. cit.; Schmoll et al., Regulation of glucose-6-phosphatase gene expression by protein kinase Ba and the forkhead transcription factor FKHR. Evidence for insulin response unit-dependent and -independent effects of insulin on promoter activity, J. Biol. Chem. 275: 36324-36333(2000); Rhee et al., Regulation of hepatic fasting response by PPARγ coactivator-1alpha (PGC-1): requirement for hepatocyte nuclear factor 4alpha in gluconeogenesis, Proc. Natl. Acad. Sci. USA 100: 4012-4017 (2003); Schmoll et al., Identification of a cAMP response element within the glucose-6-phosphatase hydrolytic subunit gene promoter which is involved in the transcriptional regulation by cAMP and glucocorticoids in H4IIE hepatoma cells, Biochem. J. 338 (Pt 2):457-463 (1999)).

Recently, an in vitro test for insulin glargine, which measures pIR in CHO-IR cells was reported by Tennagels et al. (Stimuli to the Revision Process: A Proposed In Vitro Cell Based Bioassay to Include into the USP general chapter Insulin Assays <121>, U.S. Pharmacopoeial Forum 43(4) (2017)). This assay has demonstrated a similar result as the rabbit blood sugar method in a comparison study (Ibid.), and the data were submitted to FDA and gained regulatory approval to replace rabbit blood sugar method with the pIR in vitro assay (Hack et al., Progress towards the replacement of the rabbit blood sugar bioidentity assay by an in vitro test for batch release of insulins in quality control, Altex 34(4): 565-566 (2017)).

Based upon the pIR assay, a similar pIR assay was developed using Meso Scale Discovery (MSD) technology. The MSD assay provides a format that is more convenient than the traditional enzyme-linked immunosorbent assay (ELISA) such as that disclosed by Tennagels et al. (op. cit.) because it uses a preformatted assay kit and the primary detection antibodies have been coated on the bottom of the wells of the MSD assay plate by the manufacturer. However, even though it is simpler than a traditional ELISA assay, the inventors found the MSD assay to be labor intensive with several wash steps and to display variability in results.

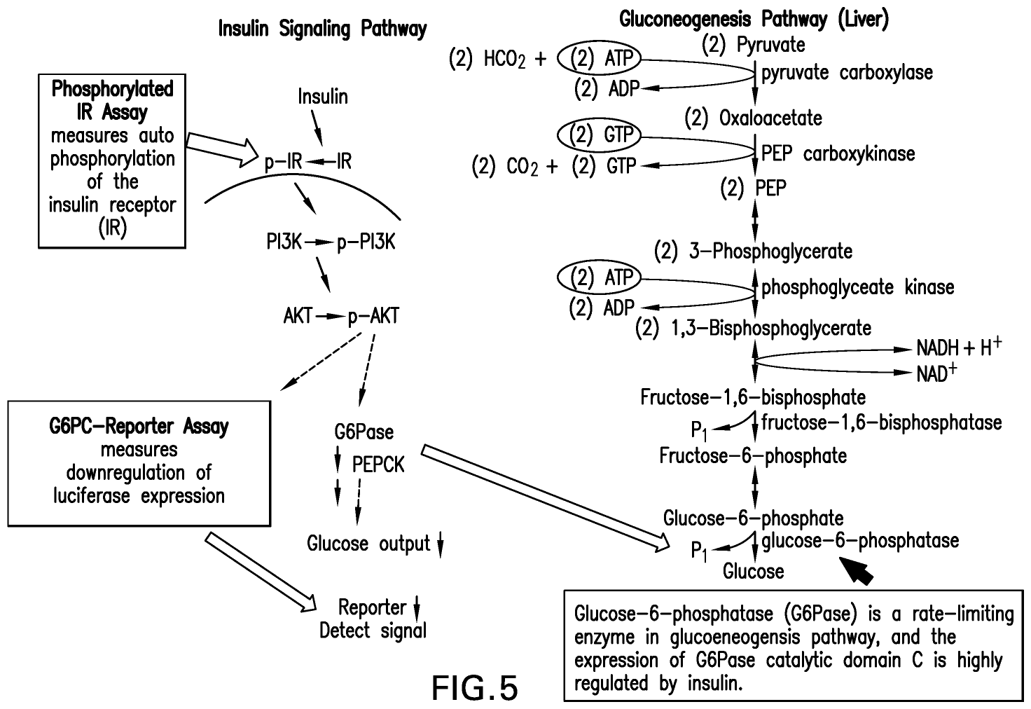

The inventors invented the present invention, a physiologically relevant functional cell-based assay that is robust and easy to run in a quality control laboratory, avoids the limitation of the pIR assay, may be used to replace the rabbit blood sugar bioidentity assay, and may also be used as a quantitative potency assay. The functional cell-based assay is an in vitro assay for determining the bioidentity or potency of insulin and insulin analogs by measuring expression of a reporter gene under the control of a glucose-6-phosphate catalytic-subunit-encoding gene C (G6PC) promoter transfected into a recombinant host cell comprising a gluconeogenesis pathway following contact of a culture of the cells with an insulin or insulin analog. In cells comprising a gluconeogenesis pathway (See FIG. 5), the G6PC promoter is downregulated by insulin; therefore, the assay measures the decrease in expression of the reporter gene when the host cell is exposed to insulin. The decrease in expression may be measured using a detectable substrate for the reporter molecule encoded by the reporter gene. For example, the reporter gene may encode an enzyme such as luciferase and expression of the luciferase may be detected by providing a luciferin substrate to the cell culture that fluoresces when cleaved by luciferase. In the assay, the bioidentity or potency of the insulin or insulin analog is inversely proportional to the decrease in expression of the reporter gene. Unlike the in vitro pIR assay disclosed in the USP<121> revision, the assay does not require use of antibodies and the washes they entail for detecting the presence and accumulation of a molecule in response to treatment with the insulin or insulin analog.

A central element of the present invention is use of a host cell capable of gluconeogenesis which comprises a nucleic acid molecule encoding a reporter molecule transcription unit operably linked to and under the regulatory control of the promoter for the glucose-6-phosphate catalytic-subunit-encoding gene C (G6PC). The resulting G6PC promoter-reporter molecule transcription unit is subject to the same regulatory control as the endogenous gene encoding the G6PC gene. Introduction of the nucleic acid molecule comprising the G6PC promoter-reporter molecule transcription unit into the host cell may be achieved using any transfection or viral transduction method known in the art under conditions known in the art that result in the nucleic acid molecule being integrated into the genome of the host cell to provide a recombinant host cell that is stably transfected with the nucleic acid molecule. The host cell may include one or more genetic additional modifications.

Gluconeogenesis is a ubiquitous process, present in plants, animals, fungi, bacteria, and other microorganisms. In vertebrates, gluconeogenesis takes place mainly in the liver and, to a lesser extent, in the cortex of the kidneys. In ruminants, this tends to be a continuous process whereas in many other animals, the process occurs during periods of fasting, starvation, low-carbohydrate diets, or intense exercise and is inhibited by insulin. In practice, the present invention is performed in a eukaryotic host cell that includes both a functional and active gluconeogenesis pathway, which is subjectable to regulatory control by insulin. Hepatocytes have both a functional and active gluconeogenesis pathway, which is subject to regulatory control by insulin. Isolated hepatocytes tend to attenuate the expression of gluconeogenic genes during a long incubation after isolation: expression of gluconeogenic genes is markedly downregulated in the isolated hepatocytes after 24 hours of incubation in a monolayer compared with that in the whole liver. Similarly, cell lines derived from liver cancers appear to become deficient in gluconeogenesis. However, H4IIE rat hepatoma cells (CRL-1548, American Type Culture Collection (ATCC), 10801 University Blvd. Manassas, Va. 20110) or HepG2 human hepatoma cells (ATCC, HB-8065) retain an active gluconeogenesis pathway (Okamoto et al., Establishment and characterization of a novel method for evaluating gluconeogenesis using hepatic cell lines, H4IIE and HepG2, Arch. Biochem. Biophys. 491: 46-52 (2009)).

Glucose-6-phosphatase (G6Pase) is a rate-limiting enzyme in gluconeogenesis pathway and the expression of G6Pase catalytic domain C (G6PC) is highly regulated by insulin and downregulated in response to insulin (Mues et. al., Molecular Mechanisms of Metformin Action, Horm. Metab. Res. 41: 730-735 (2009)). The nucleotide sequence for the G6PC promoter that was used to exemplify the present invention is set forth in SEQ ID NO: 1. As used herein, the G6PC promoter is not limited to the nucleotide sequence set forth in SEQ ID NO: 1 but includes longer or shorter nucleotide sequences provided that activity of a promoter comprising a longer or shorter nucleotide sequence than that set forth in SEQ ID NO: 1 is regulated by insulin when in a host cell with a functional gluconeogenesis pathway.

The present invention was exemplified using a nucleic acid molecule encoding a luciferase and measuring expression of the luciferase by providing the luciferase substrate luciferin in the detection step following incubation of the recombinant cells with an insulin or insulin analog for the prescribed time period. Luciferase activity on luciferin induces a fluorescent signal that is detectable using an apparatus capable of detecting the fluorescent signal. Other reporter genes may be used in the present invention such as operably linking a nucleic acid molecule encoding a secreted alkaline phosphatase to the G6PC promoter and detecting expression of the SEAP by detecting its activity to dephosphorylate a chemiluminescent alkaline phosphatase substrate CSPD (PubChem CID No. 424756) into an unstable dioxetane anion which decomposes and emits light (Berger et al., Secreted placental alkaline phosphatase: a powerful new quantitative indicator of gene expression in eukaryotic cells, Gene. 66: 1-10 (1988): Cullen et al., Secreted placental alkaline phosphatase as a eukaryotic reporter gene. Methods. Enzymol. 216:362-368 (1992)). To simplify detection of the reporter molecule, the reporter molecule is secreted into the medium or may be displayed on the surface of the recombinant cells.

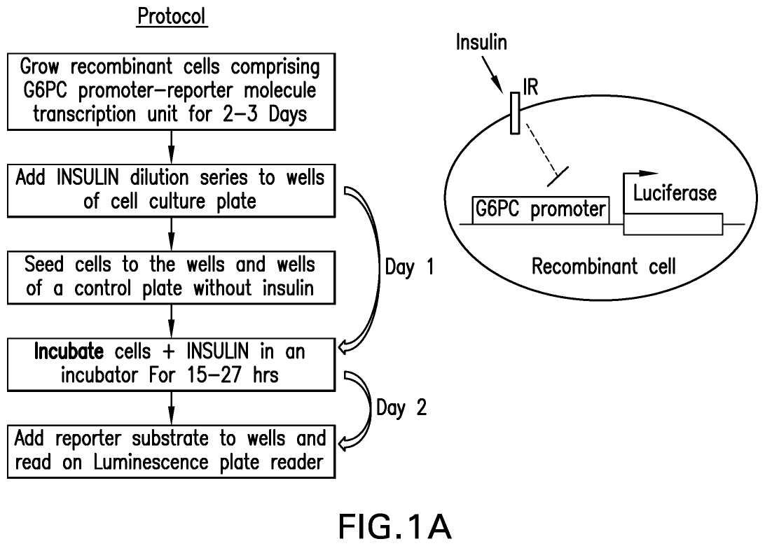

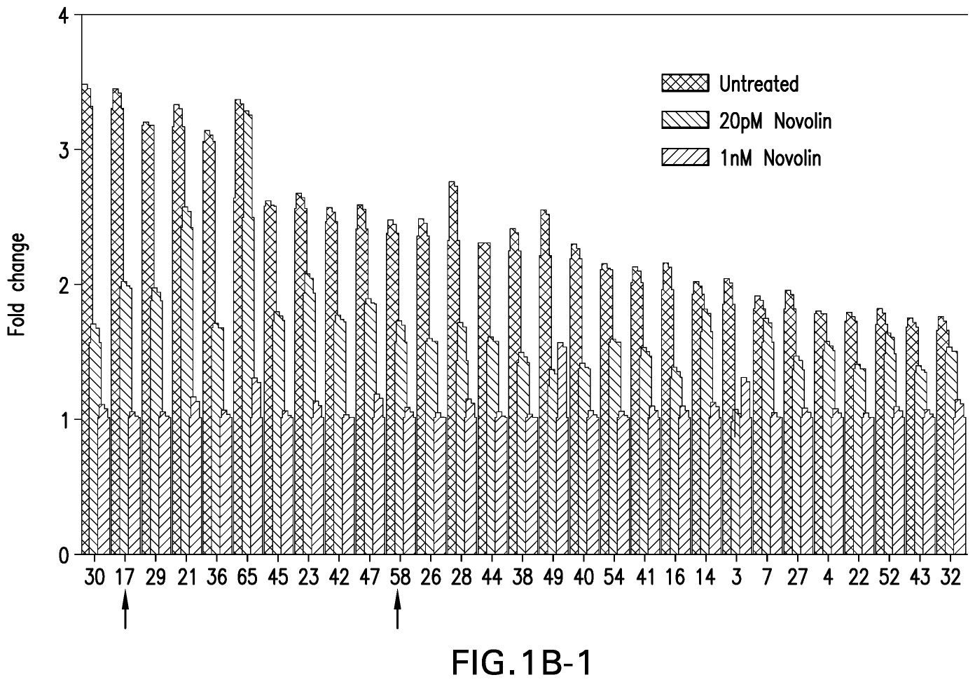

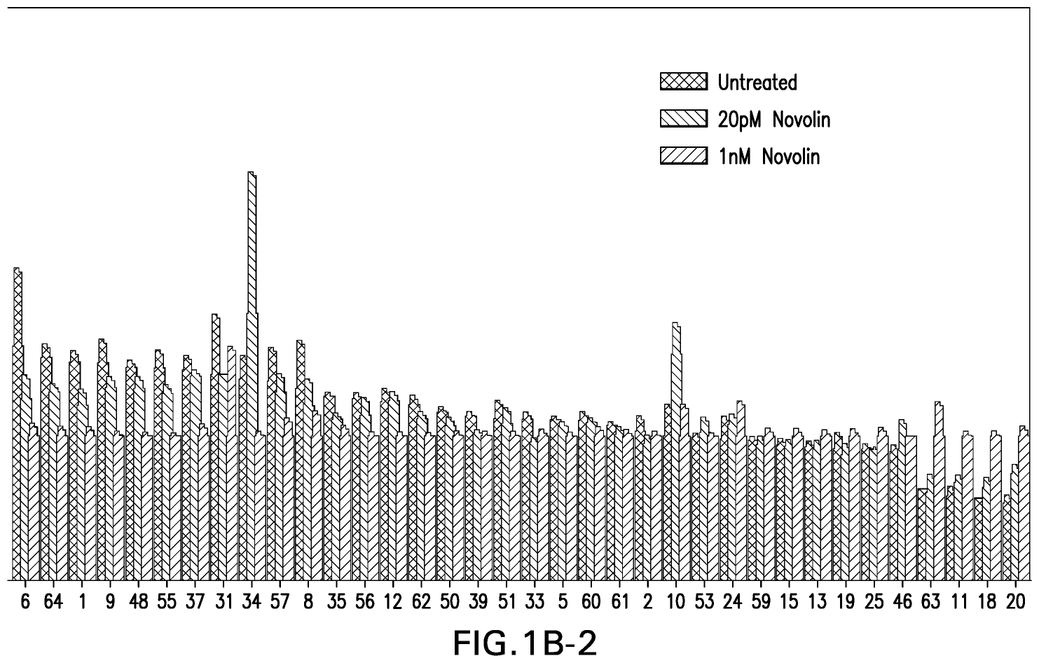

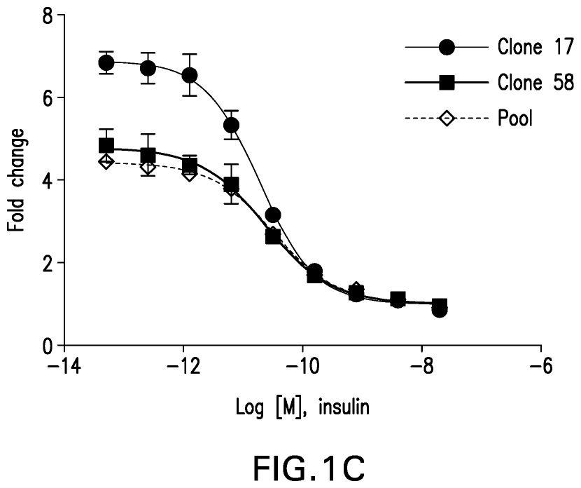

A general protocol for practicing the present invention is shown FIG. 1A. Recombinant cells capable of gluconeogenesis and comprising a nucleic acid encoding a reporter molecule operably linked to and under the regulatory control of the G6PC promoter are grown in cell culture under typical cell culturing conditions and in a typical cell culture medium, for example, Dulbecco's Modified Eagle's Medium (DMEM) supplemented with at least 5% fetal bovine serum (FBS) and at least 1.5 g/L glucose, for a time sufficient to provide the number of cells necessary for the assay. In general, the cells are cultured for two to three days in order to provide a master stock of cells comprising a sufficient quantity of cells to seed a multiplicity of wells of one or more cell culture plates at a sufficient density for performing the assay.

On the day of the assay, aliquots of a solution comprising an insulin or insulin analog in cell culture medium to be tested are dispensed into a multiplicity of wells of the one or more cell culture plates. The aliquots may be dispensed into the multiplicity of wells in a multiplicity of repeating serial dilutions.

In a typical bioidentity assay, a reference stock solution is also prepared from a reference product comprising insulin or insulin analog of a known potency by obtaining a sample solution from the reference product and diluting the sample in cell culture medium (e.g., DMEM supplemented with at least 5% FBS and at least 1.5 g/L glucose) to provide a reference solution having a predetermined concentration or potency of the insulin or insulin analog. A sample stock is prepared by obtaining a sample solution from a product batch intended for release for commercial sale and diluting the sample in cell culture medium (e.g., DMEM supplemented with at least 5% FBS and at least 1.5 g/L glucose) to provide a sample solution having a predetermined concentration of the insulin or insulin analog.

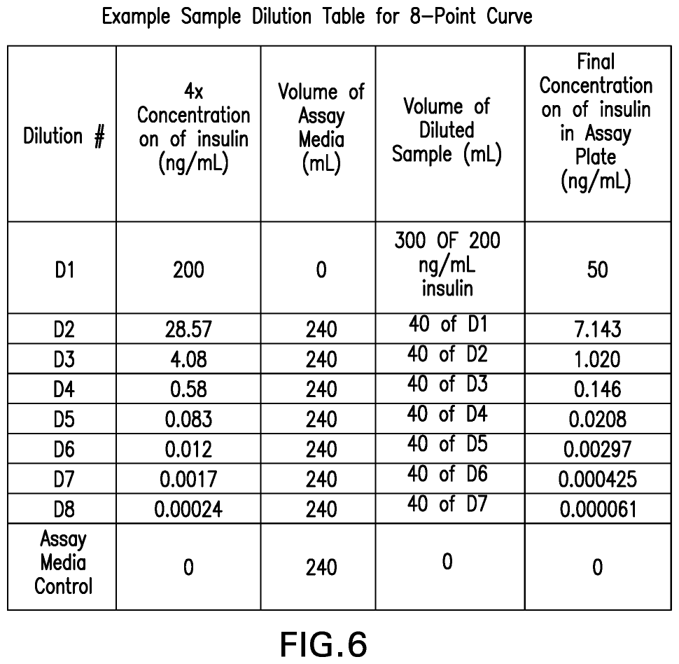

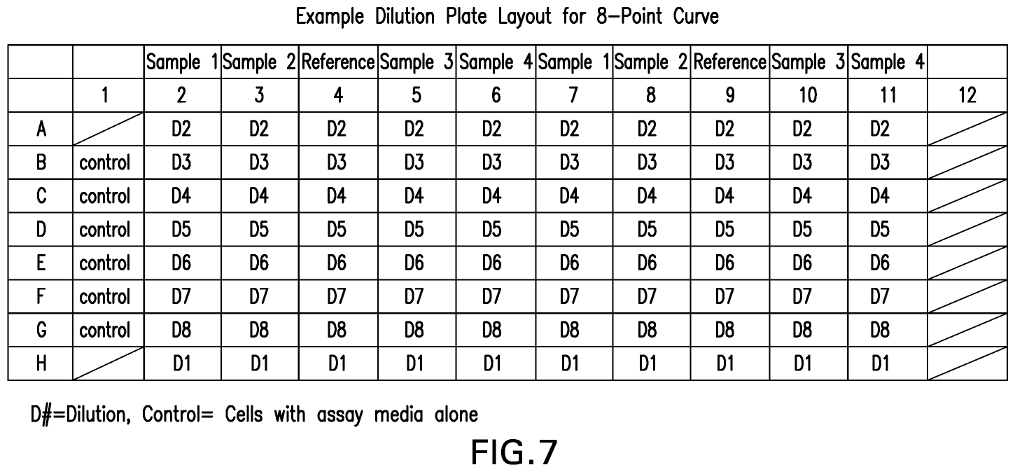

On the day of the assay, aliquots of the reference solution and the sample solution are each dispensed in serial dilution into the wells of a multiwell plate. In a typical bioidentity assay, the reference and sample solutions are dispensed into the wells in seven-fold serial dilution series to the wells of a 96-well multiwell plate. A representative example of a typical seven-fold dilution is shown in FIG. 6. The seven-fold serial dilutions along with a control that contains no insulin or insulin analog may be arranged on a 96-well plate as shown in FIG. 7.

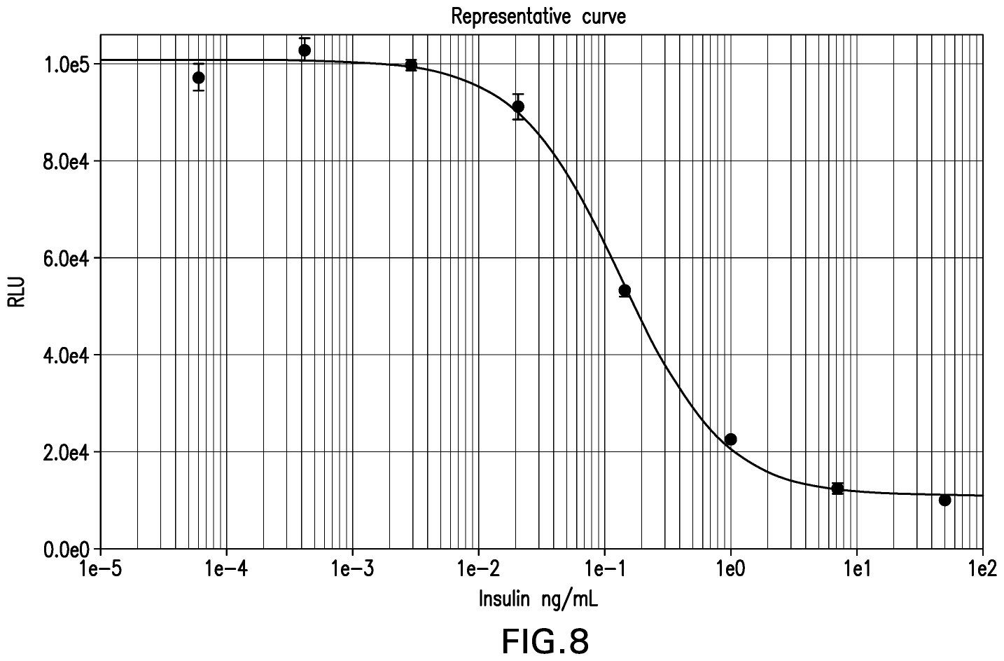

After dispensing the standard and sample dilutions, aliquots of the master stock of recombinant cells are dispensed into the wells at a density of about 20,000 to 50,000 cells/well or about 40 cells/well and the cells incubated for 15 to 27 hours at 37° C. and 5% CO₂. In a typical assay, about 17 hours provides sufficient time for the assay. Afterwards, detection of reporter molecule is performed by incubating a substrate suitable for detecting activity of the reporter molecule. When the reporter molecule is luciferase, the substrate may be luciferin, which may be detected using a fluorescence detector apparatus. The fluorescence detected for each serial dilution series is obtained and presented in an eight-point dose response curve. A representative dose response curve is shown in FIG. 8.

The insulin or insulin analog potency as measured by the assay is inversely proportional to the expression of the reporter molecule or intensity of signal from reporter molecule activity on the substrate. Therefore, the lower the fluorescence in a sample well relative to a control well containing no insulin or insulin analog, the greater the potency of the insulin or insulin analog, and a comparison of the fluorescence of a serial dilution of the sample to the fluorescence of a serial dilution of the reference provides a relative indication of the potency of the insulin or insulin in the sample. In a bioidentity test, the potency of the insulin or insulin analog in the sample is compared to the potency for the insulin or insulin analog in the reference. For a sample to have bioidentity to the reference, the relative potency of the sample should within a predetermined range of the potency for the reference. For example, the relative potency of the sample may be 100%+/−10% of the potency for the reference.

As shown in the example, comparison of a pre-qualification study using the present invention to a prequalification study using a pIR-based assay showed that present invention had less variability and a lower failure rate than the pIR-based assay, which in addition, has many steps and takes longer to perform than the present invention.

The following examples are intended to promote a further understanding of the present invention.

### Example 1

2. Material and Methods

### 2.1. Cell Line and Reagents

H4IIE rat hepatoma cell line (CRL-1548), and HepG2 (HB-8065) human hepatoma cell lines were purchased from ATCC. Both H4IIE and HepG2 cell lines are cultured in Dulbecco's Modified Eagle Medium (DMEM), supplemented with 10% fetal bovine serum (FBS) and 100 unit/mL of penicillin and 100 μg/mL of streptomycin (1× penicillin-streptomycin), during regular maintenance. DMEM, FBS, penicillin-streptomycin, phosphate buffered saline (PBS), Hygromycin B were all purchased from Gibco, a division of Thermo-Fisher Scientific. ONE-GLO luciferase assay system was purchased from Promega Corporation. Basal insulin (NOVOLIN) was purchased from Novo Nordisk. Insulin glargine (MK-1293) was made internally at Merck.

### 2.2. Generation of the H4IIE G6P-Luc C17-2 Stable Cell Line

The H4IIE G6P-Luc C17-2 stable cell line was generated by stably expressing Luciferase reporter gene under the control of G6PC promoter via lentivirus technology. Briefly, the human G6PC promoter −1227/+57 sequence (SEQ ID NO: 1) was synthesized and subcloned into pLenti-NFAT-Luc vector to replace NFAT promoter. Lentivirus was generated comprising the construct and H4IIE parental cells were transduced with the lentivirus and screened with the selection antibiotic Hygromycin B. The resulting stable pool cells were validated by quantitative polymerase chain reaction (qPCR) and luciferase assay. The initial work of generating a H4IIE G6P-Luc stable cell line was outsourced to GenScript, Piscataway, N.J. USA. The stable cell pool was re-cloned in-house with limiting dilutions to identify additional clones. H4IIE_G6P-Luc C17-2 is a single clone stable cell line selected for its high assay window (difference in fluorescence detected in a sample containing 1 nM insulin versus fluorescence detected in a sample containing no insulin) and fast growth rate.

### 2.3. H4IIE G6P-Luc Reporter Assay

The H4IIE_G6P-Luc_C17-2 cells were cultured in Dulbecco's Modified Eagle's medium (DMEM), supplemented with 10% fetal bovine serum (FBS), 1× penicillin-streptomycin, and 0.3 mg/mL Hygromycin B. On day of assay, insulin dilution was prepared in assay medium (DMEM with 10% FBS, 1× penicillin-streptomycin, and 25 mM HEPES) in a 96-well round-bottom intermediate plate at four times the final concentration. 25 μL insulin titration was aliquoted into the wells of a solid white 96-well tissue-culture treated assay plate. Immediately after adding drug to the assay plate, 75 μL H4IIE_G6P-Luc_C17-2 cells were seeded to the assay plate at a density of 40,000 cells per well and the plate incubated for 17 hours. Afterwards, 100 mL/well of ONE-GLO luciferase regent was added to the assay plate. After 10 minutes incubation at room temperature, luminescence signal was read on the EnVision plate reader (Perkin Elmer, Waltham, Mass. USA).

2.4 Comparator Phosphor-Insulin Receptor (pIR) Meso Scale Discovery (MSD) Assay

The pIR MSD assay is a sandwich immunoassay that measures phosphorylated insulin receptor using MSD technology, which may be obtained commercially as a kit from MSD. MSD provides a plate that has been pre-coated with capture antibodies for total IR on a distinct spot. The pIR is detected with an anti-phosphotyrosine antibody conjugated with an electrochemiluminescent compound, MSD SULFO-TAG label. The general assay procedure is listed in Table 6. Briefly, HepG2 cells were cultured in DMEM with 10% FBS and 1× penicillin-streptomycin. On the day before treatment, 100 μL HepG2 cells per well were seeded in a 96-well tissue-culture plate at a density of 60,000 cells per well and incubated overnight. On the second day, growth medium was removed and replaced with starvation medium (DMEM without FBS or penicillin-streptomycin). After a two-hour starvation, cells were treated with insulin for six to nine minutes. The media was removed and 30 μL lysis buffer per well was added. The cells were then incubated on ice for 35-45 minutes. Then, 25 μL of cell lysate was then transferred to the MSD assay plate for measuring pIR according to manufacturer's recommendation. The electrochemiluminescence signal was read in an MSD SECTOR Imager.

### 2.5. Pre-Qualification Design

The main objective of the pre-qualification study was to estimate the assay accuracy, intermediate precision, and linearity across the normal operating range of the assay conditions following the methods described in USP<1033> Biological Assay Verification, U.S. Pharmacopoeia (2010). All analyses were based on the natural logarithmic transformation on the relative potency values. Geometric mean, percent relative bias, percent geometric standard deviation (% GSD), and percent relative standard deviation (% RSD) were calculated using formulas from USP <1033>. All statistical analysis was carried out using JMP version 13 software (SAS Institute, Cary, N.C.).

3. Results

### 3.1. Generation of H4IIE G6P-Luc Stable Cell Line

To exemplify the functional cell-based assay of the present invention for measuring insulin activity, we generated a H4IIE G6P-Luc stable cell line that stably expressed a luciferase reporter gene under the control of a G6PC promoter (FIG. 1A). A stable pool of cells was generated, which was re-subcloned with limiting dilution and a total of 65 clones were picked. Those 65 clones were analyzed in the luciferase assay using three conditions: untreated, or treated with 20 pM or 1 nM NOVOLIN (recombinant human insulin) (FIG. 1B). Ten clones with the highest fold changes from untreated to 1 nM recombinant human insulin in the luciferase activity were selected and expanded. These ten clones were then retested with a defined cell number per well in the assay plate (data not shown). Clones 17 and 58 (C17 and C58, respectively) were selected for further expansion. These two clones were retested to compare with stable pool with more recombinant human insulin dose titrations to obtain full response curve. Clone 17 was selected because this clone had the largest assay window (FIG. 1C) and a fast growth rate. The final chosen clone, C17-2, is a derivative of C17. As used herein, the term “assay window” refers to the difference between signal detected in a positive control versus the signal detected from a negative control.

### 3.2. H4IIE G6P-Luc Reporter Assay Optimization

The reporter assay was then optimized with the C17-2. In the original screening a seven-hour treatment with recombinant human insulin was conducted. It was observed that the longer the treatment, the greater the inhibition. Therefore, in a comparison of the top two clones, an overnight recombinant human insulin treatment time was used, which resulted in significantly better inhibition (FIG. 1C).

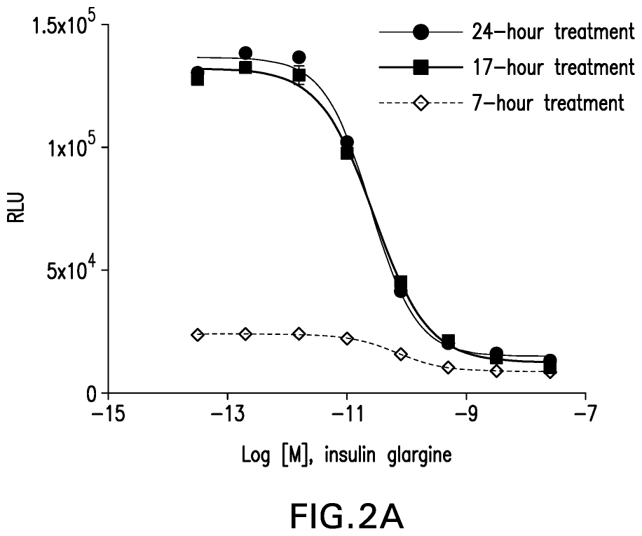

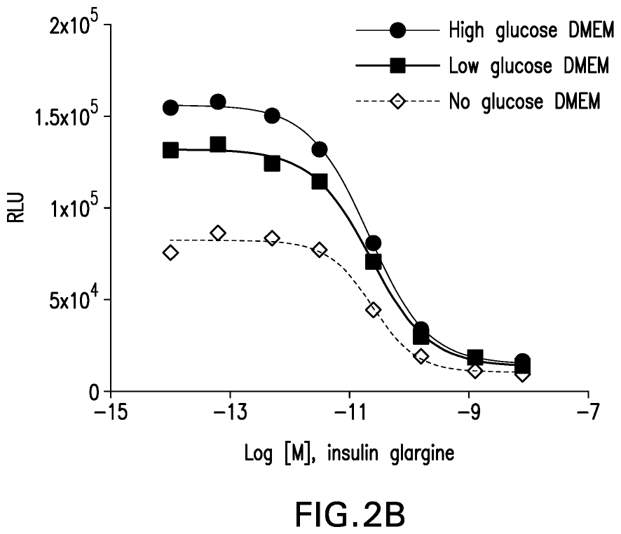

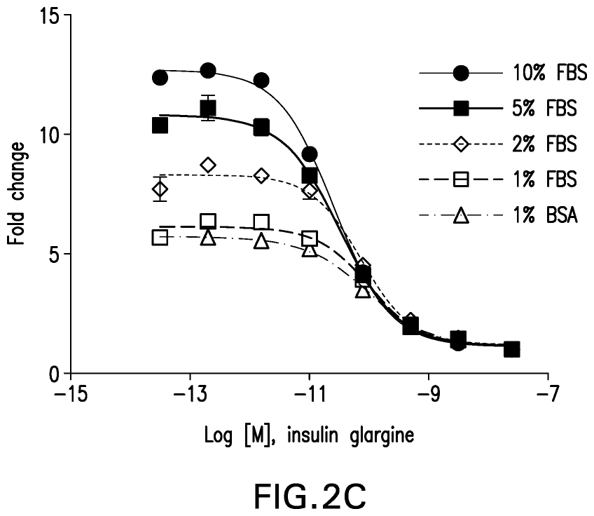

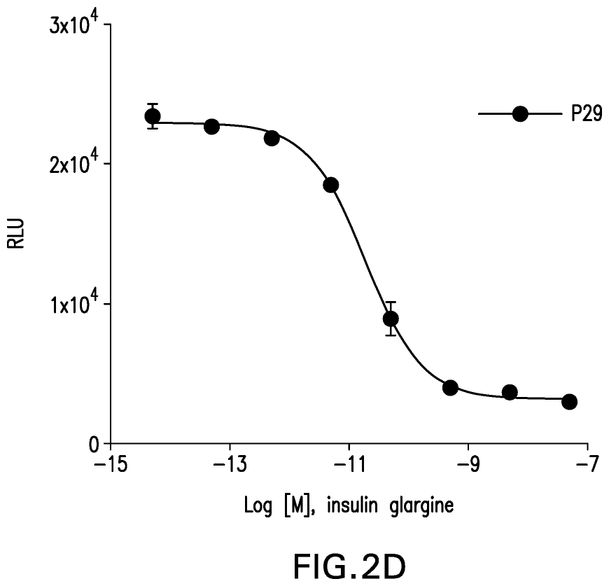

We investigated 7-hour, 17-hour, and 24-hour insulin treatment times using the final picked clone C17-2 and the recombinant human insulin analog, insulin glargine. The same results were obtained as had been observed earlier using recombinant human insulin. Only mild inhibition by insulin glargine was observed with the 7-hour treatment (FIG. 2A) whereas an overnight treatment (17-hours) resulted in robust inhibition by the insulin glargine. A 24-hour treatment gave a similar degree of inhibition as was observed for the 17-hour treatment (FIG. 2A). Since longer incubation times might begin to introduce higher variation into the method, 17-hour treatment time was selected, which provides a convenient overnight treatment option.

We next investigated whether the glucose level in the assay medium would have an impact on assay performance vel non. Since this assay implicates the gluconeogenesis pathway, it was thought that a higher glucose level might reduce gluconeogenesis activity in the cells and thus, causing a lower luciferase readout. Surprisingly, the high glucose (4.5 g/L) containing DMEM in the assay buffer provided the highest readout and the best assay window compared to DMEM with either low glucose (1.5 g/L) or no glucose (FIG. 2B). This unexpected finding might be because the cells are at their healthiest when a high amount of glucose is present in medium.

We further investigated whether fetal bovine serum (FBS) or 1% bovine serum albumin (BSA) in the assay medium had an effect on the assay. Replacing serum with 1% BSA gave the highest readout but with a lower assay window (FIG. 2C) and the cells clumped together and had a stressed appearance under this condition. Less inhibition of luciferase expression was observed with insulin glargine when lower amounts of FBS were used. Higher levels of FBS provided a higher assay window.

In addition, the clone C17-2 appears to be very stable. After 29 cell passages, a well-formed dose response curve by insulin glargine was still observed (FIG. 2D). The optimized and final conditions after additional optimization are summarized in the Table 1.

TABLE 1
Summary of assay optimization

| Parameters | Current Setting | Note |
|---|---|---|
| Cell density | 40,000 cells/75 μL/well | Can tolerate ± 10,000 |
| | | cells/well |
| Cell | Up to P29 | Minimal impact by cell |
| Passage # | | passage |
| Drug treatment | 17 ± 1 hours | 17 hour provides a good assay |
| time | | window and low variation |
| Plate type | Solid white plates | Highest signal observed with |
| | | solid white plate |
| Assay medium | High glucose DMEM | Better than low-glucose/ |
| (DMEM) | | no-glucose DMEM |
| Assay medium | 10% FBS | Lower FBS may also be used |
| additive | | |
| Plate reader | Perkin Elmer EnVision | Need sensitive plate |
| | or Molecular Devices | reader due to low |
| | SpectraMax L | luminescence signal |

### 3.3. Glargine Cell-Based Assay Pre-Qualification

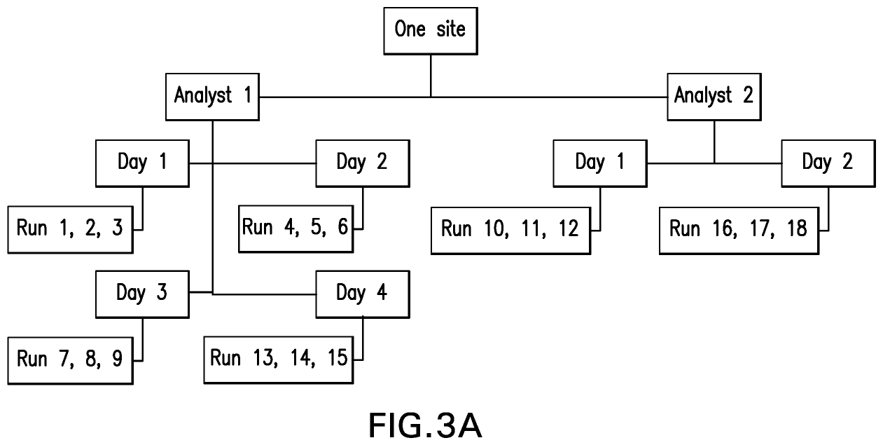

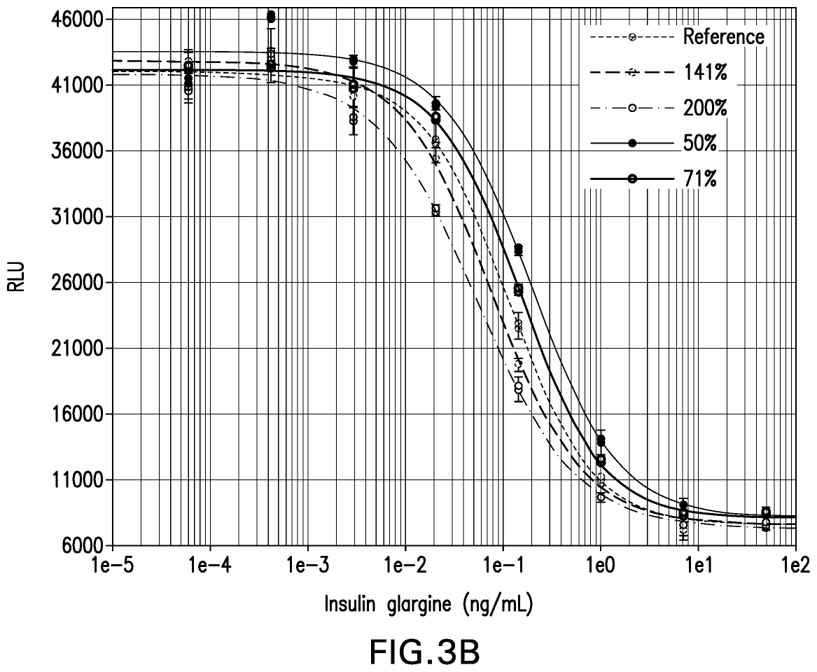

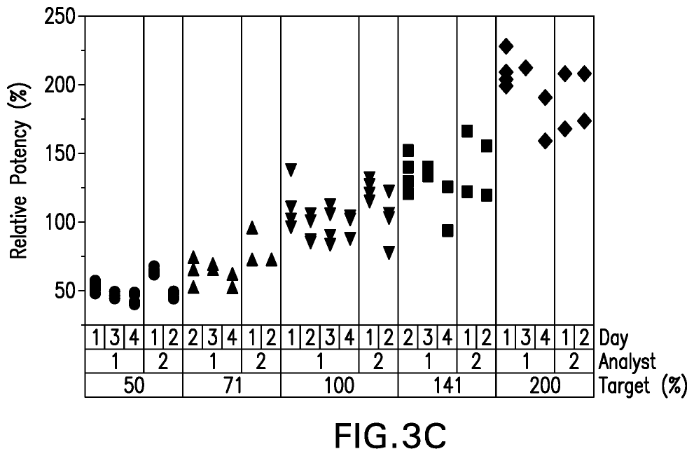

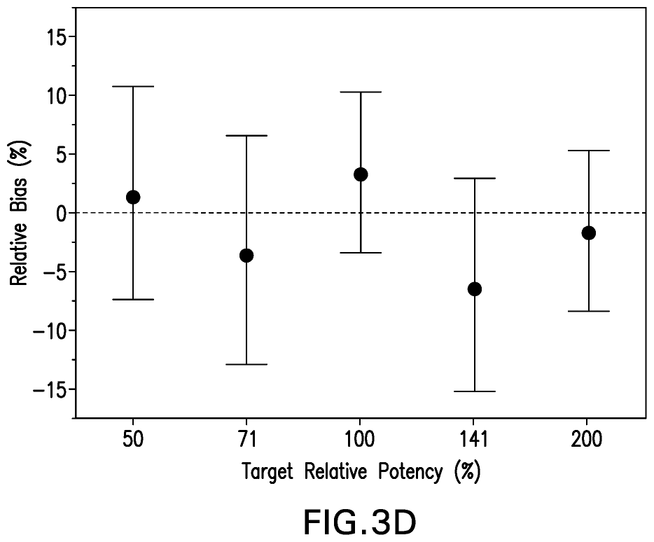

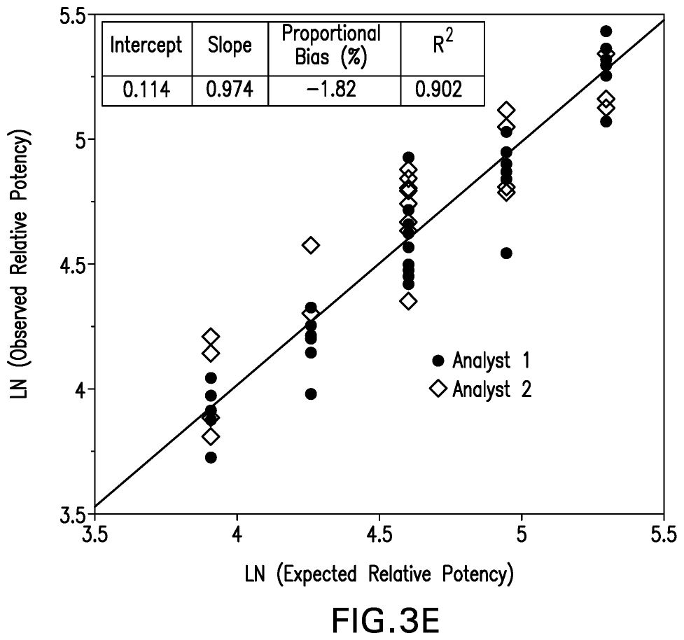

A pre-qualification study of this cell-based assay was performed to assess the following performance characteristics of the method: relative accuracy, precision, linearity and range. A pre-qualification study is similar to a qualification study except that it is performed in a non-GMP laboratory. Five potency doses were tested at a range of 50% to 200% of insulin glargine reference material (50%, 71%, 100%, 141%, and 200% relative potency levels) in a total of 18 plates. Each potency dose was tested by two analysts (lab technicians), with four independent runs (days) by one analyst and two independent runs (days) by the other analyst (FIG. 3A). 1-4 independent replicates of the same dilution were performed in each run. Since the 100% target potency is the most important value and can be used to evaluate intraplate precision, higher repeat (N=24 compared to N=12 for other potency levels) was performed. Also, insulin glargine drug substance/drug product samples will be tested at this concentration, making it particularly relevant. A representative graph of the dose-response curve results in the pre-qualification study is shown in FIG. 3B. All the relative potency data points, grouped by day, analyst and target potency, were plotted in FIG. 3C.

### 3.3.1. Relative Accuracy

Relative Accuracy, expressed as Relative bias, between the target relative potency of the dilution sample and the measured relative potency (geometric mean (GM) of relative potency (RP) of replicate samples) was calculated at individual levels of the dilutional linearity experiment using the formula:

% Relative bias=100*(relative potency measured/target relative potency−1) (Eq. 1)

The relative bias plot is shown in FIG. 3D and summarized in Table 2. Within the testing range of 50% and 200%, all measured geometric means of the five potency levels have good agreement with their target relative potency with % relative bias ranging from −6.5% to 3.2%, with recovery rate ranges from 93% to 103% respectively.

TABLE 2
Summary of assay accuracy and bias of the pre-qualification study

| Target | | | | | |
|---|---|---|---|---|---|
| Relative | | Geometric | % Relative | 95% Confidence Limits on | |
| Potency (%) | N | mean | Bias | % Relative Bias | |
| 50 | 12 | 51 | 1.3 | −7.4 | 10.8 |
| 71 | 12 | 68 | −3.7 | −12.9 | 6.6 |
| 100 | 24 | 103 | 3.2 | −3.4 | 10.3 |
| 141 | 12 | 132 | −6.5 | −15.1 | 2.9 |
| 200 | 12 | 196 | −1.8 | −8.4 | 5.3 |
| Overall | 72 | N/A | −0.8 | −3.5 | 2.1 |

### 3.3.2 Linearity and Range of Reliable Response

Linearity refers to the assays' ability to generate proportional results. This can be achieved through the calculation of proportional bias, which is related to the slope (β) from the regression of log (relative potency) on log (target potency) (Coffey et al., Biological assay qualification using design of experiments. BioProcess International 11: 42-49 (2013)). The formula is given in Equation 2.

% Proportional Bias=100×(2^{β-1}−1) (Eq. 2)

For assessing linearity, target potency values (based on dilution of insulin glargine reference material) were plotted against measured relative potency values (relative potency values for individual replicates or Geometric mean of relative potency) on a natural log scale. Regression analysis was performed and the coefficient of determination R², y-intercept, slope and the regression line are reported in FIG. 3E. As shown in FIG. 3E, the regression line generated within the tested range of 50% to 200% relative potency has a slope of 0.97 with a proportional bias of −1.8. In this case, it implies that the estimated 2-fold decrease in observed potency is about 1.8% less than what is expected for a perfectly linear assay. The data suggests that there is a good linear relationship within the tested range. Combined with relative accuracy data on each dilution level, the results indicate that the data within the tested range of 50% to 200% is reliable in this assay.

### 3.3.3. Intermediate Precision

Intermediate precision (IP) is the overall variability from analysts, days, and plates. In evaluating precision, the relative potencies were first log-transformed to satisfy the requirements of normality and variance homogeneity. Variance component analysis was carried out on the log-transformed relative potencies by performing a mixed-model analysis with restricted maximum likelihood estimation (REML) using JMP 13.0 software (SAS Institute, Cary, N.C.). Precision estimates were determined for each target potency level separately as well as overall. In the latter analysis, to account for the systematic effect due to testing samples with different potencies, the potency level was treated as a fixed linear covariate in the model. The variance estimates (s²) are converted back to the original units and expressed in terms of % RSD (relative standard deviation) and % GSD (geometric standard deviation) using the following formulas given in Equation 3.

% RSD=100×\sqrt{e^{s}^{²}−1}; % GSD=100×(e^{s}−1) (Eq. 3)

The estimated % RSD and % GSD of the variance component analyses are summarized in Table 3. The overall percent geometric standard deviation (% GSD, intermediate precision) for a target concentration of 100% was about 17% and the % GSD pooled across different concentration levels was about 16%. In all the potency levels, most of the variability in the relative potency came from within plate factors.

TABLE 3
Intermediate Precision result of the pre-qualification study

| Target | | | | | | | | |
|---|---|---|---|---|---|---|---|---|
| Relative | % RSD | | | | % GSD | | | |
| Potency | | | | Total | | | | Total |
| (%) | Analyst | Day | Residual* | IP | Analyst | Day | Residual* | IP |
| 50 | 3.4 | 14.14 | 7.0 | 16.2 | 3.4 | 15.1 | 7.2 | 17.4 |
| 71 | 13.5 | 0 | 12.9 | 18.8 | 14.4 | 0 | 13.7 | 20.5 |
| 100 | 5.2 | 6.7 | 13.1 | 15.6 | 5.3 | 6.9 | 13.9 | 16.8 |
| 141 | 0 | 0 | 15.2 | 15.2 | 0 | 0 | 16.3 | 16.3 |
| 200 | 0 | 5.5 | 9.7 | 11.2 | 0 | 5.6 | 10.2 | 11.8 |
| Overall | 4.3 | 7.7 | 12.2 | 15.1 | 4.3 | 8.0 | 12.9 | 16.2 |

*Includes the contributions from variability due to sample preparation, replicate plates, and plate position.

### 3.3.4 Format Variability

Variance component estimates were used to establish suitable assay format with desired level of precision. The predicted variability fork independent runs, with n individual dilution series of the sample preparation within a run:

Format variability=100(e^{\sqrt{Var(Run)/k+Var(Error)/n-k}}−1) (Eq. 4)

Using intra-run and inter-run variability calculation from intermediate precision estimates, the predicted variability of reportable value for various combinations of number of runs and replicates is shown in Table 4. For example, for 1 run with 3 replicates, the predicted % GSD is around 11.5%, and 2 runs with 2 replicates per run would have predicted % GSD around 8.8%. Both scenarios are common practice in the testing laboratory, and the predicted % GSD is low for a cell-based assay in either case.

TABLE 4
Format variability

| | | Variability estimates in | | | Original scale | |
|---|---|---|---|---|---|---|
| | | natural L scale | | | Predicted | Predicted |
| | | | | Reportable | % | % |
| Run | Replicate | Run | Residual | Value | RSD | GSD |
| 1 | 1 | 0.007 | 0.015 | 0.022 | 14.8 | 15.9 |
| 1 | 2 | 0.007 | 0.015 | 0.014 | 12.0 | 12.7 |
| 1 | 3 | 0.007 | 0.015 | 0.012 | 10.9 | 11.5 |
| 2 | 1 | 0.007 | 0.015 | 0.011 | 10.4 | 11.0 |
| 2 | 2 | 0.007 | 0.015 | 0.007 | 8.5 | 8.8 |
| 2 | 3 | 0.007 | 0.015 | 0.006 | 7.7 | 8.0 |

### 3.3.5 Assay Performance Acceptance Criteria

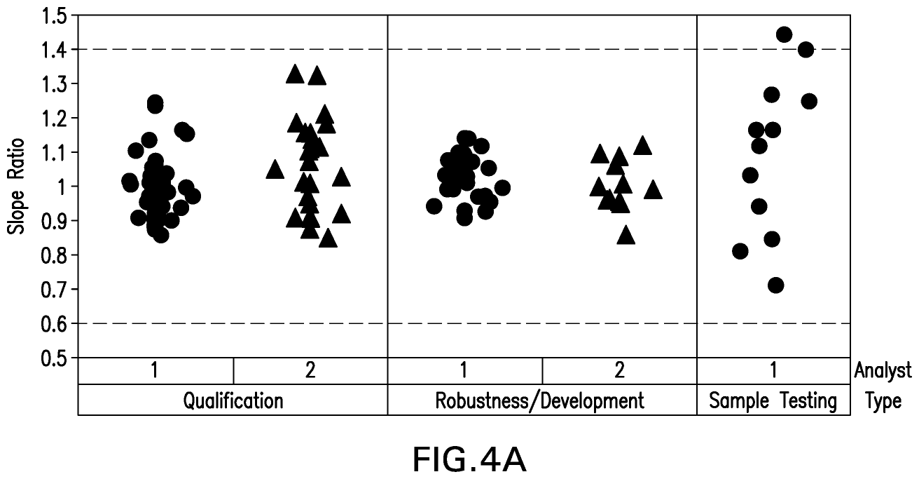

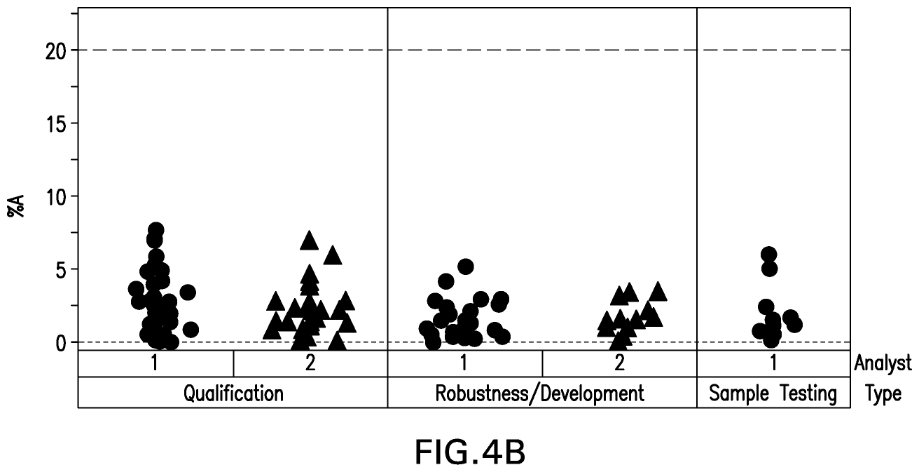

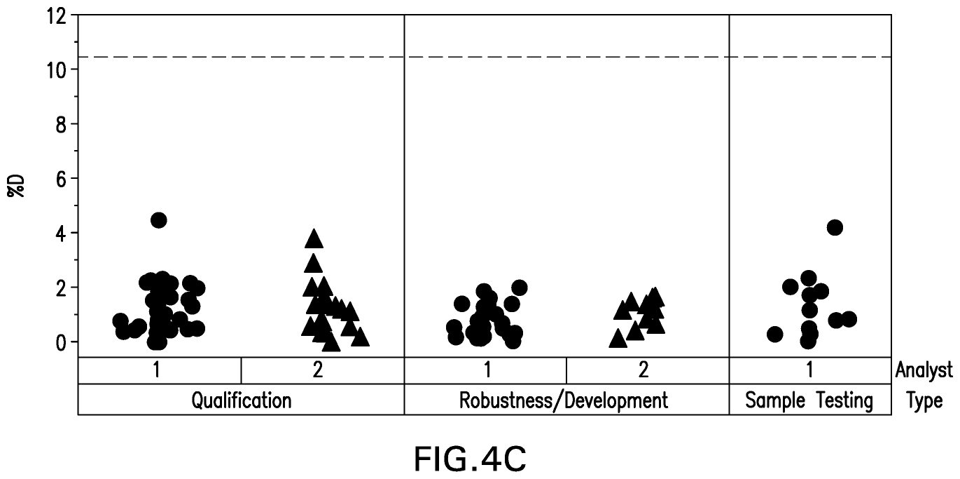

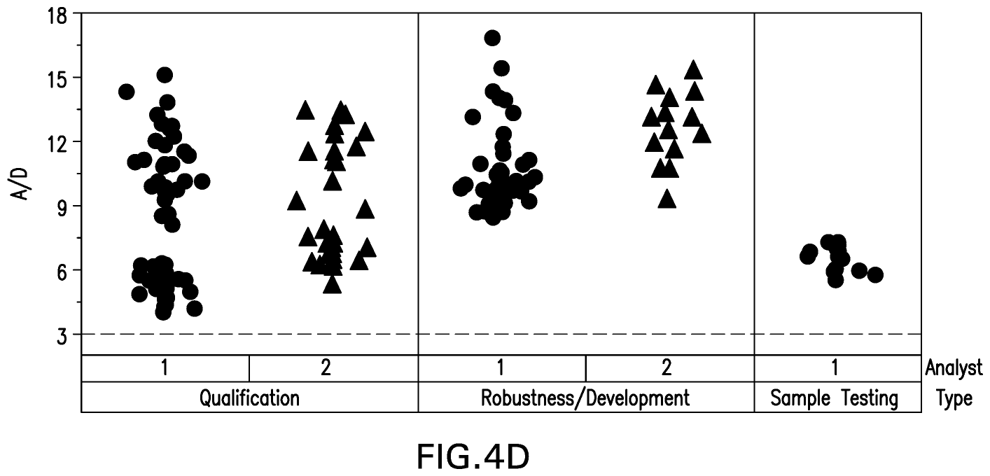

This assay did not have any pre-defined acceptance criteria in place to evaluate a valid relative potency measure for sample testing. Based on the pre-qualification, robustness/development and sample testing data, statistical acceptance criteria were proposed for system suitability parameters for the reference material and the samples, as well as sample acceptance criteria, i.e. parallelism assessment criteria between the reference and the sample curves as shown in Table 5. The acceptance criteria were also checked graphically against all the available data in FIG. 4. All system suitability parameters fell within the current acceptance limits.

TABLE 5
Acceptance Criteria for Sample Testing
and Estimates of Intermediate Precision

| | | Statistical | Current Acceptance |
|---|---|---|---|
| | | Acceptance | Limits used for |
| Purpose | Parameter | Limits | Sample Testing |
| Parallelism | % A* (upper | ≤8 | <20 |
| | % D ** (lower | <5 | <10 |
| | Slope ratio | [0.7, 1.4] | [0.6, 1.4] |
| System | A/D ratio | ≥4 | ≥3 |
| Suitability | | | |
| Intermediate | % GSD | 16% | ≤25% |
| Precision | | | |

3.4 Comparison with Current Available Phosphor-Insulin Receptor Cell-Based Assay

Recently, an in vitro test for insulin glargine was reported (Tennagels et al., Stimuli to the Revision Process: A Proposed In Vitro Cell Based Bioassay to Include into the USP general chapter Insulin Assays <121>. U.S. Pharmacopoeial Forum 43(4), (2017)), which measures insulin receptor phosphorylation (pIR) induced by insulin glargine in a Chinese hamster ovary cell line overexpressing the human insulin receptor (CHO-IR). In light of the report, a pIR assay was established, which measures insulin receptor phosphorylation in HepG2 cell line using Meso Scale Discovery (MSD) technology. The MSD pIR assay has been used to qualify Good Manufacturing Practice (GMP) standard as a non-GMP extended characterization method.

The exemplary H4IIE G6P-Luc reporter assay was compared to the MSD pIR assay by performing a pre-qualification study with the pIR MSD assay using a testing strategy similar to the H4IIE G6P-Luc pre-qualification study and comparing the results to the results obtained for the exemplary H4IIE G6P-Luc reporter assay. Compared to pIR MSD assay, the H4IIE G6P-Luc assay has significantly lower variability with intermediate precision (% GSD) at 16% compared with 21% of the pIR MSD assay (Table 6). In addition, the exemplary H4IIE G6P-Luc assay also offers many other advantages, including that the assay is easier to run in a quality control laboratory with fewer steps and without any wash steps, it is more cost effective, and without reliability on a single technology. The complete comparison of both assays is listed in Table 6.

TABLE 6
Comparison with a previously established pIR cell-based assay

| Available | | pIR MSD | | H4IIE G6P-Luc reporter |
|---|---|---|---|---|
| assays | | assay | | assay |
| Assay | Day 1: | | Day 1 | |
| procedure | 1. | Cell seeding | 1. | Drug dilution; |
| | Day 2 | | 2. | Cell seeding; |
| | 1. | Serum starve cells; | 3. | Incubate |
| | 2. | Block MSD plate; | Day 2 | |
| | 3. | Drug dilution and treatment; | 1. | Add luciferase |
| | 4. | Incubate; | | substrate; |
| | 5. | Cell lysis; | 2. | Read plate |
| | 6. | Wash MSD plate 3×; | | |
| | 7. | Add lysate to MSD plate; | | |
| | 8. | Incubate; | | |
| | 9. | Detection Ab preparation; | | |
| | 10. | Wash plate 3×; | | |
| | 11. | Add detection Ab; | | |
| | 12. | Incubate; | | |
| | 13. | Prepare read buffer; | | |
| | 14. | Wash plate; | | |
| | 15. | Add read buffer; | | |
| | 16. | Read plate. | | |
| Assay | 21% | | 16% | |
| variability | | | | |
| (intermediate | | | | |
| precision, % | | | | |
| GSD) | | | | |
| Cost | 1. | MSD plate and reagents are | 1. | Luciferase substrate is |
| | | expensive | | only a fraction cost of |
| | 2. | Labor intensive operation | | MSD plate (about 1/5 |
| | 3. | High failure rate (up to 30-40%) | | cost) |
| | | | 2. | Low labor intensity |
| | | | 3. | Rarely seen any failure |
| | | | | so far |
| Critical reagent | 1. | MSD plate and antibodies in the | 1. | Cell line is the only |
| | | assay are critical reagents. | | critical reagent (already |
| | 2. | High variability from lot to lot on | | generated in house) |
| | | MSD plates | | |
| Other | | MSD is the sole provider of MSD plate | | Not locked into a single |
| considerations | | assay reagent, and MSD plate reader | | technology |

4. Discussion

The development of an exemplary functional cell-based assay for use as a bioidentity assay to replace the rabbit blood sugar method has been described. Increased blood glucose levels in diabetic patients are primarily due to increased liver glucose production of which the gluconeogenesis pathway plays the major role in glucose production (Petersen et al., Mechanisms of Insulin Action and Insulin Resistance, Physiol. Reviews 98: 2133-2223(2018)), and G6Pase is the rate-limiting enzyme in the final step of gluconeogenesis pathway and is the primary target of insulin: its expression is dramatically downregulated by insulin thus, contributing to the glucose lowering effect in diabetic patients (ibid. and Hating et al., Insulin regulation of gluconeogenesis, Ann. NY Acad. of Sci. 1411: 21-35(2018)). Importantly, the present invention (and the exemplary H4IIE G6P-Luc cell-based assay) is highly physiologically relevant. Therefore, the insulin activity as determined using the present invention accurately reflects in vivo efficacy. In addition, the present invention is simple to perform as it does not require any wash steps since it is measuring a decrease in reporter expression in response to insulin. The assay is also economical to run because of minimal critical reagent requirements and a short analyst handling time.

The goal for an optimal assay is to achieve the best accuracy and precision, not the largest assay window. One lesson we learned during this assay development is that the larger assay window does not necessarily translate into better accuracy and precision. For example, during assay development, we also explored a longer incubation time. We observed the largest assay window when cells were incubated with insulin for 26 hours in a multiple-factorial design-of-experiment (DOE) study (data not shown). Interestingly, when we used this condition for pre-qualification, we observed higher assay variations than the final assay condition chosen, with intermediate precision around 18% (data not shown). For this functional cell-based assay, it is clear that larger assay window does not translate into lower assay variation. It is possible that longer cell incubation time may have introduced higher assay variation. There is a tradeoff between the incubation time and assay window, as we know that longer cell incubation times can introduce more variation due to heterogeneous cell growth and response to drug from well to well. The 17-hour incubation time we chose in this assay format ensures sufficient incubation time for a large assay window but also gives the analyst convenient and flexible time to plan their assay and day around.

In this exemplary assay, the H4IIE rat hepatoma cell line expresses the rat insulin receptor; however, the insulin signaling pathway is highly conserved across species and rodents have been used for more than half a century to study insulin action in vivo. Sequence alignments show that the rat insulin receptor (NCBI protein accession number NP_058767.2) shares 97% homology and 96% identity with the human insulin receptor, and the primary insulin binding site (735-743 of rat insulin receptor) is identical to the primary insulin binding site of human insulin receptor. H4IIE cells were selected because of their well-preserved gluconeogenesis pathway and insulin action (Rhee et al., Regulation of hepatic fasting response by PPARgamma coactivator-1alpha (PGC-1): requirement for hepatocyte nuclear factor 4alpha in gluconeogenesis, Proc. Natl. Acad. of Sci. USA 100: 4012-4017 (2003); Granner et al., Inhibition of transcription of the phosphoenolpyruvate carboxykinase gene by insulin, Nature 305: 549-551(1983)), as well as fast cell growth. In conclusion, the H4IIE rat cell line for the bioidentity test is expected to translate well for measuring insulin efficacy for humans, similar to the currently used rabbit blood sugar method.

Table of Sequences

| SEQ | | |
|---|---|---|
| ID | Descrip- | |
| NO: | tion | Sequence |
| 1 | human | gagctcaggaattcaagaccagcctgggcaacat |
| | G6PC | ggaaaaaccccatctctacaaaagatagaaaaat |
| | promoter | tagccaggcatggtggcgtgtgcctgtggtccca |
| | −1227/+57 | gctactcaggaggctgaggtgggaggatcacatt |
| | | agcccaggaggttgaggctgcagtgagccgtgat |
| | | tatgccactgcactccagcctgggagacagagtg |
| | | agaccctgtttcaaaaaaaagagagagaaaattt |
| | | aaaaaagaaaacaacaccaagggctgtaacttta |
| | | aggtcattaaatgaattaatcactgcattcaaaa |
| | | acgattactttctggccctaagagacatgaggcc |
| | | aataccaggaagggggttgatctcccaaaccaga |
| | | ggcagaccctagactctaatacagttaaggaaag |
| | | accagcaagatgatagtccccaatacaatagaag |
| | | ttactatattttatttgttgtttttcttttgttt |
| | | tgttttgttttgttttgttttgttttagagactg |
| | | gggtcttgctcgattgcccaggctgtagtgcagc |
| | | ggtgggacaatagctcactgcagactccaactcc |
| | | tgggctcaagcaatcctcctgcctcagcctcctg |
| | | aatagctgggactacaagggtacaccatcacaca |
| | | caccaaaacaattttttaaatttttgtgtagaaa |
| | | cgagggtcttgctttgttgcccaggctggtctcc |
| | | aactcctggcttcaagggatcctcccacctcagc |
| | | ctcccaaattgctgggattacaggtgtgagccac |
| | | cacaaccagccagaactttactaattttaaaatt |
| | | aagaacttaaaacttgaatagctagagcaccaag |
| | | atttttctttgtccccaaataagtgcagttgcag |
| | | gcatagaaaatctgacatctttgcaagaatcatc |
| | | gtggatgtagactctgtcctgtgtctctggcctg |
| | | gtttcggggaccaggagggcagacccttgcactg |
| | | ccaagaagcatgccaaagttaatcattggccctg |
| | | ctgagtacatggccgatcaggctgtttttgtgtg |
| | | cctgtttttctattttacgtaaatcaccctgaac |
| | | atgtttgcatcaacctactggtgatgcacctttg |
| | | atcaatacattttagacaaacgtggtttttgagt |
| | | ccaaagatcagggctgggttgacctgaatactgg |
| | | atacagggcatataaaacaggggcaaggcacaga |
| | | ctcatagcagagcaatcaccaccaagcctggaat |
| | | aactgcaagggctctgctgacatcttc |

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. A method for determining the potency of an insulin or insulin analog in a sample comprising:
(a) providing a cell culture of recombinant cells capable of gluconeogenesis, wherein the recombinant cells comprise a nucleic acid molecule encoding a reporter molecule operably linked to the promoter for the glucose-6-phosphate catalytic-subunit-encoding gene C (G6PC);
(b) mixing equal aliquots of the cell culture with a series of dilutions of cell culture medium comprising the insulin or insulin analog to provide a series of assay cell cultures and incubating the series of assay cell cultures for about 15 to 27 hours;
(c) detecting the reporter molecule in the series of assay cell cultures to provide data for the assay cell culture and generating a dose response curve from the data; and
(d) comparing the dose response curve to a reference dose response curve obtained from one or more series of reference assay cell cultures determined by a method comprising
(i) mixing equal aliquots of the cell culture with a series of dilutions of a reference cell culture medium comprising a known amount of the insulin or insulin analog to provide a series of reference assay cell cultures,
(ii) incubating the series of reference assay cell cultures for about 15 to 27 hours, and
(iii) detecting the reporter molecule in the series of reference assay cell cultures to provide data for the reference assay cell culture and generating the reference dose response curve from the data, to provide the potency of the insulin or insulin analog in the sample.

2. (canceled)

3. The method of claim 1, wherein the recombinant cells are H4IIE rat hepatoma cells or HepG2 human hepatoma cells, which comprise the nucleic acid molecule encoding the reporter molecule operably linked to the promoter for the glucose-6-phosphate catalytic-subunit-encoding gene C (G6PC).

4. The method of claim 1, wherein the promoter is a human G6PC promoter comprising the nucleotide sequence set forth SEQ ID NO: 1.

5. The method of claim 1, wherein the reporter molecule is an enzyme and a substrate for the enzyme is provided to the culture to detect the reporter molecule.

6. The method of claim 5, wherein the enzyme is luciferase and the substrate is luciferin.

7-9. (canceled)

10. A method for releasing a manufacturing batch or lot of drug product comprising insulin or insulin analog, comprising:
(a) providing a manufacturing batch or lot of a drug product;
(b) obtaining a sample from the batch or lot and subjecting the sample to a cell-based bioidentity test to determine whether the batch or lot can be released, wherein the bioidentity test comprises
(i) mixing equal aliquots of a cell culture of recombinant cells with a series of dilutions of cell culture medium comprising the sample to provide a series of assay cell cultures and mixing equal aliquots of the cell culture with a series of dilutions of a reference cell culture medium comprising a known amount of the insulin or insulin analog to provide a series of reference assay cell cultures;
(ii) incubating the series of assay cell cultures and the series of reference cell cultures for about 15 to 27 hours;
(iii) detecting the reporter molecule in the series of assay cell cultures to provide data for the assay cell culture and generating a sample dose response curve from the data and detecting the reporter molecule in the series of reference assay cell cultures to provide data for the reference assay cell culture and generating a reference dose response curve from the data; and
(c) comparing the sample dose response curve to the reference dose response curve and releasing the batch or lot when the difference between the sample dose response curve is within a predetermined range from the reference dose response curve;
wherein the recombinant cells capable of gluconeogenesis, wherein the recombinant cells comprise a nucleic acid molecule encoding a reporter molecule operably linked to the promoter for the glucose-6-phosphate catalytic-subunit-encoding gene C (G6PC).

11. (canceled)

12. The method of claim 10, wherein the recombinant cells are H4IIE rat hepatoma cells or HepG2 human hepatoma cells, which comprise the nucleic acid molecule encoding the reporter molecule operably linked to the promoter for the glucose-6-phosphate catalytic-subunit-encoding gene C (G6PC).

13. The method of claim 10, wherein the promoter is a human G6PC promoter comprising the nucleotide sequence set forth SEQ ID NO: 1.

14. The method of claim 10, wherein the reporter molecule is an enzyme and a substrate for the enzyme is provided to the culture to detect the reporter molecule.

15. The method of claim 14, wherein the enzyme is luciferase and the substrate is luciferin.

16-18. (canceled)

19. A provides a method for determining the bioidentity of an insulin or insulin analog in a sample comprising:
(a) providing a cell culture of recombinant cells capable of gluconeogenesis, wherein the recombinant cells comprise a nucleic acid molecule encoding a reporter molecule operably linked to the promoter for the glucose-6-phosphate catalytic-subunit-encoding gene C (G6PC);
(b) mixing equal aliquots of the cell culture with a series of dilutions of cell culture medium comprising the insulin or insulin analog to provide a series of assay cell cultures and incubating the series of assay cell cultures for about 15 to 27 hours;
(c) detecting the reporter molecule in the series of assay cell cultures to provide data for the assay cell culture and generating a dose response curve from the data; and
(d) comparing the dose response curve to a reference dose response curve obtained from one or more series of reference assay cell cultures determined by a method comprising
(i) mixing equal aliquots of the cell culture with a series of dilutions of a reference cell culture medium comprising a known amount of the insulin or insulin analog to provide a series of reference assay cell cultures,
(ii) incubating the series of reference assay cell cultures for about 15 to 27 hours, and
(iii) detecting the reporter molecule in the series of reference assay cell cultures to provide data for the reference assay cell culture and generating the reference dose response curve from the data, to provide the bioidentity of the insulin or insulin analog in the sample.

20-28. (canceled)

29. A recombinant cell capable of gluconeogenesis comprising a nucleic acid molecule encoding a reporter molecule operably linked to the promoter for the glucose-6-phosphate catalytic-subunit-encoding gene C (G6PC).

30. The recombinant cell of claim 29, wherein the recombinant cells are H4IIE rat hepatoma cells or HepG2 human hepatoma cells.

31-33. (canceled)

34. A cell culture comprising a medium and a recombinant cell capable of gluconeogenesis comprising a nucleic acid molecule encoding a reporter molecule operably linked to the promoter for the glucose-6-phosphate catalytic-subunit-encoding gene C (G6PC).

35. The cell culture of claim 34, wherein the recombinant cells are H4IIE rat hepatoma cells or HepG2 human hepatoma cells.

36-41. (canceled)

42. A nucleic acid molecule encoding a reporter molecule operably linked to the promoter for the glucose-6-phosphate catalytic-subunit-encoding gene C (G6PC).

43-45. (canceled)

46. A vector comprising the nucleic acid molecule of claim 42.

47. The vector of claim 46, wherein the vector is a plasmid or recombinant virus.

48. The vector of claim 46, wherein the recombinant virus is a lentivirus.
